(19) United States
(12) Patent Application Publication
(10) Pub. No.: US 2016/0101148 A1
(43) Pub. Date: Apr. 14, 2016

(54) TREATMENT AND PREVENTION OF BACTERIAL SKIN INFECTIONS USING ORITAVANCIN

(71) Applicant: THE MEDICINES COMPANY, Parsippany, NJ (US)

(72) Inventors: Greg MOECK, Saint Laurent (CA); Theresa MATKOVITS, West Orange, NJ (US); Stephan A. BILLSTEIN, Franklin Lakes, NJ (US); Gina EAGLE, Morristown, NJ (US); Ketna PATEL, Bridgewater, NJ (US); Francis F. ARHIN, Laval (CA)

(73) Assignee: THE MEDICINES COMPANY, Parsippany, NJ (US)

(21) Appl. No.: 14/786,106

(22) PCT Filed: Apr. 15, 2014
(86) PCT No.: PCT/US14/34083

§ 371 Date: Oct. 21, 2015

Related U.S. Application Data
(60) Provisional application No. 61/841,368, filed on Jun. 30, 2013.
(60) Provisional application No. 61/814,577, filed on Apr. 22, 2013.

(51) Int. Cl. A61K 38/14; A61K 9/00

(52) U.S. Cl. CPC A61K 38/14; A61K 9/0019

(57) ABSTRACT

Methods for the treatment and prevention of bacterial skin infections using the glycopeptide antibiotic oritavancin are disclosed.

### TECHNICAL FIELD

The present invention relates to treatment and prevention of bacterial skin infections using the glycopeptide antibiotic oritavancin.

### BACKGROUND

Methicillin-resistant Staphylococcus aureus (MRSA) continues to predominate as causative pathogen in acute bacterial skin and skin structure infections (ABSSSI) despite a 20% decline in the incidence of invasive MRSA infections in the U.S. over the last 4 to 5 years (CDC 2011; Hadler et al. 2012). The overall disease and economic burden remains substantial (Landrum et al. 2012), with annual costs of community-associated (CA) MRSA infections estimated at up to $2.2 billion on third-party payers; the key driver of these costs is hospitalization (Jenkins et al. 2010; Lee et al. 2012). For ABSSSI patients in the current era of CA-MRSA, hospitalization is frequent and total duration of therapy often exceeds 7 to 10 days (Jenkins et al. 2010).

Besides the safety, monitoring and resistance issues that affect linezolid, daptomycin, and ceftaroline, these and other approved MRSA agents require once or twice daily dosing for at least 7 to 10 days (Stevens et al. 2005; Liu et al. 2011; prescribing information: Cubicin, Teflaro, Tygacil, Vancomycin, Vibativ, Zyvox). Hence, even if care can be provided in an outpatient setting, current ABSSSI treatments suffer from the high cost and inconvenience of multiple administrations, from incomplete medication adherence (Ball et al. 2010) and from complexity of monitoring for laboratory abnormalities outside the hospital setting. Ensuring patient compliance and reducing both hospital admissions and use of ambulatory care resources could improve health outcomes and benefit hospital systems and payers by improving ABSSSI treatment effectiveness.

Alternative means for treating, and even preventing, bacterial skin infections are urgently needed. The present invention is directed to this need and other important goals.

### BRIEF SUMMARY

The present invention relates to methods of treating and preventing bacterial skin infections in a subject. Exemplary infections include complicated skin and skin structure infections (cSSSI), acute bacterial skin and skin structure infections (ABSSSI), complicated and uncomplicated skin and soft tissue infections (SSTI), wound infections, cellulitis, abscesses, and skin lesions. The methods of the invention generally comprise administering a therapeutically effective amount of a pharmaceutical composition comprising oritavancin or a pharmaceutically acceptable salt thereof to a subject having a bacterial skin infection or to a subject at risk of developing a bacterial skin infection, thereby treating or preventing a bacterial skin infection in a subject.

In a first aspect, the invention is drawn to methods of treating or preventing a bacterial skin infection in a subject, comprising administering a therapeutically effective amount of a pharmaceutical composition comprising oritavancin or a pharmaceutically acceptable salt thereof to a subject having a bacterial skin infection or at risk of developing a bacterial skin infection, thereby treating or preventing a bacterial skin infection in a subject.

In this aspect, the infection may be, but is not limited to, one or more of a complicated skin and skin structure infection (cSSSI), a complicated and uncomplicated skin and soft tissue infection (SSTI), a wound infection, a burn infection, cellulitis, an abscess, and a skin lesion. In one embodiment, cSSSI is an acute bacterial skin and skin structure infection (ABSSSI).

In some embodiments of this aspect, the treatment achieves a cessation of an increase in surface area of the infection within about 24 hours of the administering. In additional embodiments of this aspect, the treatment achieves a prevention in increase in the surface area of the skin infection within 24 hours of the administering. In other embodiments of this aspect, the treatment achieves a reduction in surface area of the infection of at least about 20% within about 48 hours of the administering. In further embodiments of this aspect, the treatment achieves a reduction in fever in the subject within about 12 hours of the administering.

In some embodiments of this aspect, the pharmaceutical composition comprises at least about 1200 mg of oritavancin or a salt thereof.

In some embodiments of this aspect, treatment or prevention is achieved by administering to the subject a single dose of a pharmaceutical composition comprising about 1200 mg of oritavancin or a salt thereof.

In a second aspect, the invention is drawn to methods of treating or preventing a wound infection in a subject, comprising administering a therapeutically effective amount of a pharmaceutical composition comprising oritavancin or a pharmaceutically acceptable salt thereof to a subject having a wound infection or at risk of developing a wound infection, thereby treating or preventing a wound infection in a subject.

In some embodiments of this aspect, the treatment achieves a cessation of an increase in surface area of the infection within about 24 hours of the administering. In additional embodiments of this aspect, the treatment achieves a prevention in increase in the surface area of the skin infection within 24 hours of the administering. In other embodiments of this aspect, the treatment achieves a reduction in surface area of the infection of at least about 20% within about 48 hours of the administering. In further embodiments of this aspect, the treatment achieves a reduction in fever in the subject within about 12 hours of the administering.

In some embodiments of this aspect, the pharmaceutical composition comprises at least about 1200 mg of oritavancin or a salt thereof.

In some embodiments of this aspect, treatment or prevention is achieved by administering to the subject a single dose of a pharmaceutical composition comprising about 1200 mg of oritavancin or a salt thereof.

In some embodiments of this aspect, the wound is an open wound. In other embodiments, the wound is a closed wound. Examples of open wounds include, but are not limited to, incisions, lacerations, abrasions, punctures, penetration wounds, and gunshots. Examples of closed wounds include, but are not limited to, contusions, hematomas, and crush injuries.

In a third aspect, the invention is drawn to methods of treating or preventing cellulitis in a subject, comprising administering a therapeutically effective amount of a pharmaceutical composition comprising oritavancin or a pharmaceutically acceptable salt thereof to a subject having cellulitis or at risk of developing cellulitis, thereby treating or preventing cellulitis in a subject.

In some embodiments of this aspect, the treatment achieves a cessation of an increase in surface area of cellulitis within about 24 hours of the administering. In additional embodiments of this aspect, the treatment achieves a prevention in increase in the surface area of cellulitis within 24 hours of the administering. In other embodiments of this aspect, the treatment achieves a reduction in surface area of cellulitis of at least about 20% within about 48 hours of the administering. In further embodiments of this aspect, the treatment achieves a reduction in fever in the subject within about 12 hours of the administering.

In some embodiments of this aspect, the pharmaceutical composition comprises at least about 1200 mg of oritavancin or a salt thereof.

In some embodiments of this aspect, treatment or prevention is achieved by administering to the subject a single dose of a pharmaceutical composition comprising about 1200 mg of oritavancin or a salt thereof.

In a fourth aspect, the invention is drawn to methods of treating or preventing an abscess in a subject, comprising administering a therapeutically effective amount of a pharmaceutical composition comprising oritavancin or a pharmaceutically acceptable salt thereof to a subject having an abscess or at risk of developing an abscess, thereby treating or preventing an abscess in a subject.

In some embodiments of this aspect, the treatment achieves a cessation of an increase in surface area of the abscess within about 24 hours of the administering. In additional embodiments of this aspect, the treatment achieves a prevention in increase in the surface area of the abscess within 24 hours of the administering. In other embodiments of this aspect, the treatment achieves a reduction in surface area of the abscess of at least about 20% within about 48 hours of the administering. In further embodiments of this aspect, the treatment achieves a reduction in fever in the subject within about 12 hours of the administering.

In some embodiments of this aspect, the pharmaceutical composition comprises at least about 1200 mg of oritavancin or a salt thereof.

In some embodiments of this aspect, treatment or prevention is achieved by administering to the subject a single dose of a pharmaceutical composition comprising about 1200 mg of oritavancin or a salt thereof.

In this aspect, the abscess may be, but is not limited to, a skin abscess, an internal abscess, a dental abscess, a brain abscess, Bartholin's abscess, a liver abscess, a spinal cord abscess, an anorectal abscess, and a peritonsillar abscess.

In a fifth aspect, the invention is drawn to methods of treating or preventing a skin lesion in a subject, comprising administering a therapeutically effective amount of a pharmaceutical composition comprising oritavancin or a pharmaceutically acceptable salt thereof to a subject having a skin lesion or at risk of developing a skin lesion, thereby treating or preventing a skin lesion in a subject.

In some embodiments of this aspect, the treatment achieves a cessation of an increase in surface area of the lesion within about 24 hours of the administering. In additional embodiments of this aspect, the treatment achieves a prevention in increase in the surface area of the lesion within 24 hours of the administering. In other embodiments of this aspect, the treatment achieves a reduction in surface area of the lesion of at least about 20% within about 48 hours of the administering. In further embodiments of this aspect, the treatment achieves a reduction in fever in the subject within about 12 hours of the administering.

In some embodiments of this aspect, the pharmaceutical composition comprises at least about 1200 mg of oritavancin or a salt thereof.

In some embodiments of this aspect, treatment or prevention is achieved by administering to the subject a single dose of a pharmaceutical composition comprising about 1200 mg of oritavancin or a salt thereof.

In this aspect, the skin lesion may be, but is not limited to, an ulcer, a macule, a vesicle, a pustule, a papule, a nodule, a wheal, and telangiectasia.

In a sixth aspect, the invention is drawn to methods of reducing the size of a skin lesion in a subject, comprising administering a therapeutically effective amount of a pharmaceutical composition comprising oritavancin or a pharmaceutically acceptable salt thereof to a subject having a skin lesion, thereby reducing the size of a skin lesion in a subject.

In some embodiments of this aspect, the administration achieves a reduction in surface area of the lesion within about 24 hours of the administering. In other embodiments of this aspect, the administration achieves a reduction in surface area of the lesion of at least about 20% within about 48 hours of the administering.

In some embodiments of this aspect, the pharmaceutical composition comprises at least about 1200 mg of oritavancin or a salt thereof.

In some embodiments of this aspect, reduction in size is achieved by administering to the subject a single dose of a pharmaceutical composition comprising about 1200 mg of oritavancin or a salt thereof.

In a seventh aspect, the invention is drawn to methods of accelerating reduction of a symptom of a bacterial skin infection in a subject, comprising administering a therapeutically effective amount of oritavancin or a salt thereof to a subject having a bacterial skin infection, wherein the symptom is reduced more rapidly than in a subject receiving an alternative antibiotic or no antibiotic, thereby accelerating reduction of a symptom of a bacterial skin infection in a subject.

In this aspect, the alternative antibiotic may be, but is not limited to, a glycopeptide (e.g., vancomycin, teicoplanin, dalbavancin, ramoplanin), a lipoglycopeptide (e.g., daptomycin, telavancin), an oxazolidinone (e.g., linezolid, eperezolid, posizolid, radezolid, ranbezolid, torezolid), a streptogramin (e.g., quinupristin-dalfopristin, pristinamycin, virginiamycin), a glycylcycline (e.g., tigecycline), a cephalosporin (e.g., ceftaroline), a beta-lactam (e.g., cephalexin, dicloxacillin, amoxicillin), clindamycin, trimethoprim, sulfamethoxazole, doxycycline, minocycline, tetracycline, omadacycline, gentamicin, rifampin, avarofloxacin and delafloxacin.

In this aspect, the symptom of a bacterial skin infection may be, but is not limited to, tenderness of the skin, redness, swelling, and fever. In certain embodiments of this aspect, the reduction is a reduction of fever in the subject within about 12 hours of the administering.

In some embodiments of this aspect, the pharmaceutical composition comprises at least about 1200 mg of oritavancin or a salt thereof.

In some embodiments of this aspect, the acceleration is achieved by administering to the subject a single dose of a pharmaceutical composition comprising about 1200 mg of oritavancin or a salt thereof.

In an eighth aspect, the invention is drawn to a method of treating or preventing a bacterial skin infection in a human subject, preferably an adult human subject, comprising administering a therapeutically effective amount of oritavancin or a salt thereof to a human subject having a bacterial skin infection caused by susceptible isolates of one or more Gram-positive microorganisms selected from the group consisting of Staphylococcus aureus (including methicillin-susceptible and -resistant isolates), Streptococcus pyogenes, Streptococcus agalactiae, Streptococcus anginosus group (including S. anginosus, S. intermedius, and S. constellatus), and Enterococcus faecalis.

In some embodiments of this aspect, the bacterial skin infection is a complicated skin and skin structure infection (cSSSI). In some other embodiments of this aspect, the bacterial skin infection is an acute bacterial skin and skin structure infection (ABSSSI).

In some embodiments of this aspect, the treatment achieves a cessation of an increase in surface area of the infection within about 24 hours of the administering. In additional embodiments of this aspect, the treatment achieves a prevention in increase in the surface area of the skin infection within 24 hours of the administering. In other embodiments of this aspect, the treatment achieves a reduction in surface area of the infection of at least about 20% within about 48 hours of the administering. In further embodiments of this aspect, the treatment achieves a reduction in fever in the subject within about 12 hours of the administering.

In some embodiments of this aspect, the pharmaceutical composition comprises at least about 1200 mg of oritavancin or a salt thereof.

In some embodiments of this aspect, treatment or prevention is achieved by administering to the subject a single dose of a pharmaceutical composition comprising about 1200 mg of oritavancin or a salt thereof.

In an ninth aspect, the invention is drawn to pharmaceutical compositions for use in treating or preventing a bacterial skin infection in a subject, said composition comprising at least about 1200 mg oritavancin or a pharmaceutically acceptable salt thereof.

In this aspect, the infection may be, but is not limited to, one or more of a complicated skin and skin structure infection (cSSSI), an acute bacterial skin and skin structure infection (ABSSSI), a complicated and uncomplicated skin and soft tissue infection (SSTI), a wound infection, cellulitis, a burn infection, an abscess, and a skin lesion.

In each of the aspects of the invention, the source of the infection and the bacteria causing the cellulitis, abscess or lesion, is a Gram-positive bacteria, including, but not limited to, one or more of Staphylococcus aureus, methicillin-susceptible Staphylococcus aureus (MSSA), methicillin-resistant Staphylococcus aureus (MRSA), a multi-drug resistant (MDR) strain of MSSA, a MDR strain of MRSA, a mecC-expressing strain of methicillin-resistant Staphylococcus aureus (MRSA), vancomycin-resistant Staphylococcus aureus, vancomycin-intermediate Staphylococcus aureus, vancomycin hetero-intermediate Staphylococcus aureus, Streptococcus pyogenes, Streptococcus agalactiae, Streptococcus anginosus, Streptococcus intermedius, Streptococcus constellatus, Streptococcus dysgalactiae, Streptococcus dysgalactiae subsp. equisimilis, Streptococci Group C, F and G species, Staphylococcus lugdunensis, Enterococcus faecalis, vancomycin-resistant Enterococcus faecalis, Enterococcus faecium, and vancomycin-resistant Enterococcus faecium.

In each of the aspects of the invention, the administering may be via intravenous administration.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits the invention.

### BRIEF DESCRIPTION OF DRAWINGS

FIG. 1 shows disposition and analysis sets of patients in the SOLO I Trial. Notes: MRSA=methicillin-resistant Staphylococcus aureus, ori=oritavancin, van=vancomycin. a=The intent-to-treat (ITT) population included all patients randomized into the study. b=The modified intent-to-treat (mITT) was the primary population for all the efficacy analyses and included all randomized patients who received any study drug. c=The safety population was the primary population for all the safety analyses, and consisted of all patients who were dosed with study drug, irrespective of randomization. Treatment classification was based on the actual treatment received. d=2 patients randomized to receive oritavancin were inadvertently dosed with vancomycin. e=The clinically evaluable (CE) population consisted of all mITT patients who met the inclusion/exclusion criteria, received the full-course of randomized study treatment (for a minimum of 7 days), and had investigator assessment for clinical cure at PTE. The CE population was used to confirm the efficacy analyses. f=The microbiologically ITT (MicroITT) population consisted of all mITT patients with baseline gram-positive pathogen(s) known to cause ABSSSI and it was used for the secondary efficacy analyses. g=The microbiologically evaluable (MicroE) population was used to confirm the secondary efficacy analyses and consisted of all patients who were in both the MicroITT and CE populations. h=Patients may appear in more than one category.

FIG. 2 shows the results of an in vitro time-kill assay against strain S. aureus 70611 (mecC) using oritavancin and comparators at free peak and free trough. A, ORI, oritavancin, 16 mg/L (free peak) and 2 mg/L (free trough); B, VAN, vancomycin, 16 mg/L (free peak) and 4 mg/L, free trough; C, DAP, daptomycin, 4 mg/L (free peak) and 0.5 mg/L (free trough); D, LZD, linezolid, 8 mg/L (free peak) and 2 mg/L (free trough).

### DETAILED DESCRIPTION

### I. Definitions

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

The present invention takes advantage of the excellent properties of the lipoglycopeptide oritavancin and applies them to methods of treating and preventing bacterial skin infections in subjects, as well as to compositions for use in such methods. Oritavancin exhibits rapid, concentration-dependent bactericidal activity against all clinically-relevant gram-positive pathogens. It has been discovered that the unique pharmacokinetic (PK)-pharmacodynamic (PD) profile of the drug allows for treatment of skin infections using a moderate amounts of the drug that are also safe and well-tolerated in patients.

The invention relates to methods of treating or preventing bacterial skin infections in a subject. Exemplary infections include complicated skin and skin structure infections (cSSSI), acute bacterial skin and skin structure infections (ABSSSI), complicated and uncomplicated skin and soft tissue infections (SSTI), wound infections, cellulitis, burn infections, abscesses, and skin lesions. The methods of the invention generally comprise administering a therapeutically effective amount of a pharmaceutical composition comprising oritavancin or a pharmaceutically acceptable salt thereof to a subject having a bacterial skin infection or to a subject at risk of developing a bacterial skin infection, thereby treating or preventing a bacterial skin infection in a subject.

The methods of the invention comprise contacting bacteria with an effective amount of oritavancin. For example, one can inhibit cell wall biosynthesis in a gram-positive bacterium by contacting such a bacterium with oritavancin. As used herein, the term “contacting” is meant to broadly refer to bringing a bacterial cell and a molecule of oritavancin into sufficient proximity such that oritavancin can exert an effect on the bacterial cell. Oritavancin may be transported to the location of the bacterial cell, or oritavancin may be situated in a location to which the bacterial cell travels or is brought into contact. The skilled artisan will understand that the term “contacting” includes physical interaction between oritavancin and a bacterial cell, as well as interactions that do not require physical interaction.

### Methods of Treatment and Prevention

The invention includes methods of treating or preventing bacterial skin infections in a subject. Each of the methods generally comprises administering a therapeutically effective amount of a pharmaceutical composition comprising oritavancin or a pharmaceutically acceptable salt thereof to a subject having a bacterial skin infection or at risk of developing a bacterial skin infection, thereby achieving treatment or prevention of a bacterial skin infection in a subject.

In particular aspects, the methods of the invention are drawn to methods of: (a) treating or preventing ABSSSI in a subject, (b) treating or preventing a wound infection in a subject, (c) treating or preventing cellulitis in a subject, (d) treating or preventing a burn in a subject, (e) treating or preventing an abscess in a subject, and (f) treating or preventing a skin lesion in a subject. In each of these aspects, the method comprises administering a therapeutically effective amount of a pharmaceutical composition comprising oritavancin or a pharmaceutically acceptable salt thereof to a subject having the noted bacterial skin infection or at risk of developing the noted bacterial skin infection, thereby treating or preventing the noted bacterial skin infection in a subject.

In other aspects, the invention is drawn to methods of reducing the size of a skin lesion in a subject, comprising administering a therapeutically effective amount of a pharmaceutical composition comprising oritavancin or a pharmaceutically acceptable salt thereof to a subject having a skin lesion, thereby achieving a reduction in the size of the skin lesion in a subject.

In still other aspects, the invention is drawn to methods of accelerating reduction of a symptom of a bacterial skin infection in a subject, comprising administering a therapeutically effective amount of oritavancin or a salt thereof to a subject having a bacterial skin infection, wherein the symptom is reduced more rapidly than in a subject receiving an alternative antibiotic or no antibiotic, thereby accelerating reduction of a symptom of a bacterial skin infection in a subject. In this aspect, the alternative antibiotic may be, but is not limited to, a glycopeptide (e.g., vancomycin, teicoplanin, dalbavancin, ramoplanin), a lipoglycopeptide (e.g., daptomycin, telavancin), an oxazolidinone (e.g., linezolid, eperezolid, posizolid, radezolid, ranbezolid, torezolid), a streptogramin (e.g., quinupristin-dalfopristin, pristinamycin, virginiamycin), a glycylcycline (e.g., tigecycline), a cephalosporin (e.g., ceftaroline), a beta-lactam (e.g., cephalexin, dicloxacillin, amoxicillin), clindamycin, trimethoprim, sulfamethoxazole, doxycycline, minocycline, tetracycline, omadacycline, gentamicin, rifampin, avarofloxacin and delafloxacin. In this aspect, the symptom of a bacterial skin infection may be, but is not limited to, tenderness of the skin, redness, swelling, and fever.

In yet other aspects, the invention is drawn to methods of treating or preventing a bacterial skin infection in a human subject, preferably an adult human subject, comprising administering a therapeutically effective amount of oritavancin or a salt thereof to a human subject having a bacterial skin infection caused by susceptible isolates of one or more Gram-positive microorganisms selected from the group consisting of Staphylococcus aureus (including methicillin-susceptible and -resistant isolates), Streptococcus pyogenes, Streptococcus agalactiae, Streptococcus anginosus group (including S. anginosus, S. intermedius, and S. constellatus), and Enterococcus faecalis (vancomycin-susceptible isolates only).

As used herein, “bacterial skin infection” refers to an infection of the skin, soft tissue, or both, caused by a species or strain of bacteria for which the methods disclosed herein are appropriate. For example, the methods of treatment may be used in the treatment of subjects having bacterial skin infections, such as complicated skin and skin structure infections (cSSSI), and complicated and uncomplicated skin and soft tissue infections (SSTI). Acute bacterial skin and skin structure infections (ABSSSI) are a subset of cSSSI and they are a further example of the bacterial skin infections of the present invention. More particularly, bacterial skin infections include wounds, abscesses, lesions, burns, and cellulitis, with specific examples including, but not limited to, minor cutaneous abscesses; deep bacterial infections, such as major abscess, major cutaneous abscess, infected ulcer, major burn, and deep and extensive cellulitis; animal or human bites; necrotizing fasciitis; diabetic foot infection; decubitus ulcer infection; myonecrosis, ecthyma gangrenosum; catheter-site infections; impetigo. The methods of treatment can also be practiced concomitantly with surgical intervention for the bacterial skin infection.

ABSSSI includes one or more of wound infections (either traumatic or surgical in origin), cellulitis/erysipelas, major cutaneous abscess and burn infection, often with surrounding erythema, edema, and/or induration of at least 75 cm² (see U.S. FDA Guidance for Industry-Acute Bacterial Skin and Skin Structure Infections: Developing Drugs for Treatment, at the website having the URL fda.gov/downloads/Drugs/ . . . /Guidances/ucm071185.pdf). In particular, ABSSSI includes one or more of the following infections:

A. Wound infections: either traumatic or surgical in origin defined as an infection characterized by purulent drainage from a wound with surrounding erythema, edema, and/or induration of a minimum surface area of 75 cm² (e.g., the shortest distance of erythema, edema, and/or induration extending at least 5 cm from the peripheral margin of the wound). The infection may be accompanied by one or more signs and systemic inflammation symptoms such as purulent drainage or discharge, erythema, fluctuance, heat or localized warmth, edema/induration, pain or tenderness to palpation, proximal lymph node swelling and tenderness, increased temperature (≧38.0° C.), decreased temperature (<36.0° C.), increased white blood cell count (>10,000 cells/mm³), bandemia>10%, and C-reactive protein (CRP)>upper limit of normal (ULN).

B. Cellulitis/erysipelas: a diffuse skin infection characterized by spreading areas of erythema, edema, and/or induration of a minimum surface area of 75 cm² (e.g., minimum length of 10 cm and width of 7.5 cm). The infection may be accompanied by one or more signs and systemic inflammation symptoms such as purulent drainage or discharge, erythema, fluctuance, heat or localized warmth, edema/induration, pain or tenderness to palpation, proximal lymph node swelling and tenderness, increased temperature (≧38.0° C.), decreased temperature (<36.0° C.), increased white blood cell count (>10,000 cells/mm³), bandemia>10%, and C-reactive protein (CRP)>upper limit of normal (ULN).

C. Major cutaneous abscess: an infection characterized by a collection of pus within the dermis or deeper accompanied by erythema, edema, and/or induration of a minimum surface area of 75 cm² (e.g., the shortest distance of erythema, edema, and/or induration extending at least 5 cm from the peripheral margin of the abscess). The infection may be accompanied by one or more signs and systemic inflammation symptoms such as purulent drainage or discharge, erythema, fluctuance, heat or localized warmth, edema/induration, pain or tenderness to palpation, proximal lymph node swelling and tenderness, increased temperature (≧38.0° C.), decreased temperature (<36.0° C.), increased white blood cell count (>10,000 cells/mm³), bandemia>10%, and C-reactive protein (CRP)>upper limit of normal (ULN).

D. Burn infection: an infection characterized by purulent drainage, redness, edema, and/or induration of a minimum surface area of 75 cm² (e.g., the shortest distance of redness, edema, and/or induration extending at least 5 cm from the peripheral margin of the burn infection). The infection may be accompanied by one or more signs and systemic inflammation symptoms such as purulent drainage or discharge, erythema, fluctuance, heat or localized warmth, edema/induration, pain or tenderness to palpation, proximal lymph node swelling and tenderness, increased temperature (≧38.0° C.), decreased temperature (<36.0° C.), increased white blood cell count (>10,000 cells/mm³), bandemia>10%, and C-reactive protein (CRP)>upper limit of normal (ULN).

The infectious bacteria and those bacteria causing bacterial infections that may be treated or prevented via the compositions and methods of the present invention include those described in U.S. Pat. No. 5,840,684, Gram-positive bacteria, and in particular, Staphylococcus aureus (including methicillin-susceptible and -resistant strains; vancomycin-susceptible, -intermediate, -hetero-intermediate and -resistant strains; multi-drug resistant (MDR) strains, mecC-expressing strains), Streptococcus pyogenes, Streptococcus agalactiae, Streptococcus anginosus grp. (including S. anginosus, S. intermedius, and S. constellatus), Streptococcus dysgalactiae (including S. dysgalactiae subsp. equisimilis), Streptococcus pneumoniae, Streptococci species, including Streptococci Group A species, Streptococci Group B species, Streptococci Group C species, Streptococci Group D species, Streptococci Group F and Streptococci Group G, Enterococci species, Enterococcus faecalis (vancomycin-susceptible and -resistant strains), Enterococcus faecium (vancomycin-susceptible and -resistant strains), Staphylococcus epidermidis (methicillin-susceptible and -resistant strains), Staphylococcus haemolyticus, Staphylococcus lugdunensis, Bacillus anthracis, and Clostridium difficile (both vegetative form and spores). A bacterial skin infection that is treated or prevented using the methods of the present invention may involve one, two, three, four, five, or even more of the bacterial species and strains provided above.

The wound infections that may be treated or prevented using the methods of the invention include both open wounds and closed wounds. Examples of open wounds include, but are not limited to, incisions, lacerations, abrasions, punctures, penetration wounds, and gunshots. Examples of closed wounds include, but are not limited to, contusions, hematomas, and crush injuries.

The abscesses that may be treated or prevented using the methods of the invention include, but are not limited to, skin abscesses, internal abscesses, dental abscesses, brain abscesses, Bartholin's abscess, liver abscesses, spinal cord abscesses, anorectal abscesses, and peritonsillar abscesses.

The skin lesions that may be treated or prevented using the methods of the invention include, but are not limited to, ulcers, macules, vesicles, pustules, papules, nodules, wheals, and telangiectasia.

### Methods of Treatment

The terms “treating” and “treatment” mean at least the mitigation of a disease condition or symptom associated with a bacterial infection in a subject that is achieved by a reduction of growth, replication, and/or propagation, or death or destruction of bacteria, on or in the subject. The terms “treating” and “treatment” include curing, healing, inhibiting, relieving from, improving and/or alleviating, in whole or in part, the disease condition. The mitigation of a disease condition or symptom may be about 100%, 99%, 98%, 97%, 96%, 95%, 90%, 80%, 70%, 60%, 50%, 40%, 30%, 20%, 10%, 5% or 1% in the subject, versus a subject to which oritavancin has not been administered. In one aspect, treating means reducing the population of bacteria causing the infection in the subject to an undetectable level, where detection is by any conventional means, such culturing a sample in the laboratory. In another aspect, treating means complete healing of the infection, shown by an absence of clinical symptoms associated with the infection. In further aspect of the invention, treating means the mitigation of a disease condition or symptom by at least about 90% in the subject. In an additional aspect, treating means the mitigation of a disease condition or symptom by at least about 95% in the subject.

In a particular aspects, treating means achieving a cessation of increase in skin infection surface area, whether it be an abscess, lesion, wound, cellulitis, or some other bacterial skin infection, within about 8, 10, 12, 14, 16, 18, 20, or 24 hours, or within about 0.5, 1, 1.5, 2, 2.5, 3, 3.5, 4, 4.5, 5, 5.5, 6, 6.5, 7, 7.5, 8, 8.5, or 9 days of the administration of oritavancin. In a further aspect, treating means achieving a reduction in skin infection surface area within about 8, 10, 12, 14, 16, 18, 20, or 24 hours, or within about 0.5, 1, 1.5, 2, 2.5, 3, 3.5, 4, 4.5, 5, 5.5, 6, 6.5, 7, 7.5, 8, 8.5, or 9 days of the administration of oritavancin. The surface area of the skin infection is reduced, versus the surface area of the infection at the time treatment is begun, by at least about 5%, 10%, 15%, 20%, 25%, 30%, 35%, 40%, 45%, or 50%, or more. In one aspect, the reduction in surface area of the skin infection is at least about 20% within about 12, 24, 36, 48, 60 or 72 hours of the administration of oritavancin. In another aspect, treating means achieving a reduction in fever in the subject within about 4, 6, 8, 10, 12, 14, 16, 18, 20, or 24 hours, or within about 0.5, 1, 1.5, 2, 2.5, 3, 3.5, 4, 4.5, 5, 5.5, 6, 6.5, 7, 7.5, 8, 8.5, or 9 days of the administration of oritavancin. In an additional aspect, treating means preventing an increase in the surface area of the skin infection within about 4, 6, 8, 10, 12, 14, 16, 18, 20, or 24 hours, or within about 0.5, 1, 1.5, 2, 2.5, 3, 3.5, 4, 4.5, 5, 5.5, 6, 6.5, 7, 7.5, 8, 8.5, or 9 days of the administration of oritavancin.

The therapeutically effective amount of a pharmaceutical composition comprising oritavancin and the amount sufficient to achieve the stated goals of the methods of treatment disclosed herein will vary, for example, in view of the physical characteristics of the subject, the severity of the subject's symptoms, the form of the infection, the identity of the bacteria, the formulation and the means used to administer the drug, and the method being practiced. The specific dose for a given subject is usually set by the judgment of the attending physician. However, in each dose the pharmaceutical composition typically comprises between about 100 mg and 3000 mg, between about 400 mg to about 1800 mg, between about 500 mg to about 1600 mg, between about 600 mg to about 1400 mg, between about 800 mg to about 1200 mg, between about 1000 mg to about 1400 mg, or between about 1100 mg to about 1400 mg of oritavancin.

In certain aspects, the pharmaceutical composition comprises about 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950 or 2000 mg of oritavancin. In specific aspects, the pharmaceutical composition contains about 800, 900, 1000, 1100, 1200, 1300 or 1400 mg of oritavancin.

In certain other aspects, the pharmaceutical composition comprises at least about 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950 or 2000 mg of oritavancin. In a preferred aspect, the pharmaceutical composition contains at least about 800, 900, 1000, 1100, 1200, 1300 or 1400 mg of oritavancin.

Depending on the means of administration, the dosage may be administered all at once, such as with an oral formulation in a capsule, or slowly over a period of time, such as with an intravenous administration. For slower means of administration, the administering period can be a matter of minutes, such as about 5, 10, 15, 20, 25, 30, 35, 40, 45, 50, 55, 60, 65, 70, 75, 80, 85, 90, 95, 100, 105, 110, 115, 120 or more minutes, or a period of hours, such as about 0.5, 1, 1.5, 2, 2.5, 3, 3.5, 4, 4.5, 5 or more hours. The administration of the dose may be interrupted, such as where the dose is administered via intravenous infusion and the dose is divided into two or more infusion bags. Under such circumstances, the administration of the dose may be interrupted while the infusion bags are changed.

Administration frequencies for the pharmaceutical compositions of the present invention will vary based on the method being practiced, the physical characteristics of the subject, the severity of the subject's symptoms, the form of the infection, the identity of the bacteria, and the formulation and the means used to administer the drug. However, administration frequencies will generally include 4, 3, 2 or once daily, every other day, every third day, every fourth day, every fifth day, every sixth day, once weekly, every eight days, every nine days, every ten days, bi-weekly, monthly and bi-monthly. In certain aspects, the pharmaceutical composition is administered once daily. The duration of treatment will be based on the condition being treated and will be best determined by the attending physician. Under some conditions, treatment will be continued for a number of days, weeks, or months. Under other conditions, complete treatment will be achieve through administering one, two or three dose of the pharmaceutical composition over the entire course of treatment. In certain aspects, complete treatment can be achieved using a single dose of the pharmaceutical composition. In a particular aspect, complete treatment is achieved using a single dose of a pharmaceutical composition comprising about 800, 900, 1000, 1100, 1200, 1300 or 1400 mg of oritavancin.

Each of the methods of the present invention may also be practiced by administering a second therapeutic agent to the subject. Such second therapeutic agents may be included in a pharmaceutical formulation comprising oritavancin, or they may be administered separately. A wide range of second therapeutic agents, such as antibiotics, can be used in combination with the compounds, compositions and methods of the present invention. Antibiotics used as second therapeutic agents may act by interfering with cell wall synthesis, plasma membrane integrity, nucleic acid synthesis, ribosomal function, folate synthesis, etc. A non-limiting list of useful antibiotics includes: fusidic acid, trimethoprim, sulfadiazine, sulfamethoxazole, a penicillin, a monobactam, a penam, a penem, a clavam, a clavem, a carbopenam, a carbopenem, a cepham, a cephem, an oxacepham, an oxacephem, a carbocepham, a carbocephem, a cephalosporin, tetracycline, a tetracycline derived antibacterial agent, glycylcycline, a glycylcycline derived antibacterial agent, minocycline, a minocycline derived antibacterial agent, sancycline, a sancycline derived antibacterial agent, methacycline, a methacycline derived antibacterial agent, an oxazolidinone antibacterial agent, an aminoglycoside antibacterial agent, an additional glycopeptide or lipoglycopeptide, a quinolone antibacterial agent, daptomycin, a daptomycin derived antibacterial agent, rifamycin, a rifamycin derived antibacterial agent, rifampin, a rifampin derived antibacterial agent, rifalazil, a rifalazil derived antibacterial agent, rifabutin, a rifabutin derived antibacterial agent, rifapentin, a rifapentin derived antibacterial agent, rifaximin and a rifaximin derived antibacterial agent. The second therapeutic agent may be administered before, concurrently with, or after a pharmaceutical formulation of the present invention is administered to a subject.

### Methods of Prevention

The terms “prevent” and “prevention” mean blocking or stopping a disease condition associated with a bacterial infection from developing in a subject, preferably a human. Such methods may be practiced, for example, on subjects having a higher risk for bacterial infection than the general population, including patients undergoing treatment for bacterial infections whereby normal gut flora is inhibited by antimicrobial therapy, patients with impaired immune function (e.g., immunoglobulin deficiency, splenic dysfunction, splenectomy, HIV infection, impaired leukocyte function, hemoglobinopathies), the elderly (Loo et al., 2005. NEJM 353:2442), people with certain malignancies (e. g., multiple myeloma, chronic lympocytic leukemia, lymphoma), people at increased occupational risk (e.g., public services workers, such a fire, water, sanitary, police, medical, and laboratory workers, hospital workers), people in closed populations (e.g., prisons, military, nursing homes) and others that have immunological deficiencies that might enhance their susceptibility to bacterial infection.

Many physicians believe that humans should be considered for antibiotic prophylaxis before a surgical procedure, a dental procedure or invasive medical procedure to mitigate the potential for an infection resulting from ineffective sterility during the procedure. Deep infection is a serious complication sometimes requiring subsequent medical interventions and is accompanied by significant morbidity and mortality. Oritavancin may therefore be used as a prophylactic antibiotic in this situation. For instance, oritavancin and/or pharmaceutical compositions of the invention may be administered to a subject to achieve a systemic and/or local effect against relevant bacteria shortly before an invasive medical treatment, such as surgery or insertion of an in-dwelling device (e.g. joint replacement (hip, knee, shoulder, etc.)). Treatment may be repeated after the invasive medical treatment, such as post-operatively or during the in-body time of the device.

The prevention achieved by the methods of the invention may last in the subject for at least about 0.5, 1, 2, 3, 4, 5, 6, 7, 8, 9, 10, 11, 12, 13, 14, 15, 16, 17, 18, 19, 20, 25, 30, 35, 40, 45, 50 or more days after administration of oritavancin.

In one aspect of the invention, the prevention lasts at least about 24 hours in the subject. In another aspect, the prevention lasts at least about 72 hours in the subject. In further aspect, the prevention lasts at least about 144 hours in the subject.

Oritavancin or a pharmaceutical composition comprising the compound may be administered within 10, 9, 8, 7, 6, 5, 4, 3, 2, or 1 day, or within 24, 22, 20, 18, 16, 14, 12, 10, 9, 8, 7, 6, 5, 4, 3, 2, 1 or 0.5 hour prior to when a subject will potentially be exposed to bacteria, such prior to contact by military personnel with a material suspected of containing a particular bacteria or prior to surgery.

The therapeutically effective amount of a pharmaceutical composition comprising oritavancin for prevention will vary depending, for example, upon the physical characteristics of the subject, the identity of the bacteria to which the subject may be exposed, the formulation and the means used to administer the drug. The specific dose for a given subject is usually set by the judgment of the attending physician. However, in each dose the pharmaceutical composition typically comprises between about 100 mg and 3000 mg, between about 400 mg to about 1800 mg, between about 500 mg to about 1600 mg, between about 600 mg to about 1400 mg, between about 800 mg to about 1200 mg, between about 1000 mg to about 1400 mg, or between about 1100 mg to about 1400 mg of oritavancin.

In certain aspects, the pharmaceutical composition comprises about 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950 or 2000 mg of oritavancin. In specific aspects, the pharmaceutical composition contains about 800, 900, 1000, 1100, 1200, 1300 or 1400 of mg oritavancin.

In certain other aspects, the pharmaceutical composition comprises at least about 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950 or 2000 mg of oritavancin. In a preferred aspect, the pharmaceutical composition contains at least about 800, 900, 1000, 1100, 1200, 1300 or 1400 mg of oritavancin.

Depending on the means of administration, the dosage may be administered all at once, such as with an oral formulation in a capsule, or slowly over a period of time, such as with an intravenous administration. For slower means of administration, the administering period can be a matter of minutes, such as about 5, 10, 15, 20, 25, 30, 35, 40, 45, 50, 55, 60, 65, 70, 75, 80, 85, 90, 95, 100, 105, 110, 115, 120 or more minutes, or a period of hours, such as about 0.5, 1, 1.5, 2, 2.5, 3, 3.5, 4, 4.5, 5 or more hours. The administration of the dose may be interrupted, such as where the dose is administered via intravenous infusion and the dose is divided into two or more infusion bags. Under such circumstances, the administration of the dose may be interrupted while the infusion bags are changed.

Administration frequencies for the pharmaceutical compositions of the present invention will vary based on the method being practiced, the physical characteristics of the subject, the identity of the bacteria to which the subject may be exposed, the formulation and the means used to administer the drug. However, administration frequencies will generally include 4, 3, 2 or once daily, every other day, every third day, every fourth day, every fifth day, every sixth day, once weekly, every eight days, every nine days, every ten days, bi-weekly, monthly and bi-monthly. In certain aspects, the pharmaceutical composition is administered once daily. Under some conditions, complete prevention will be achieve through administering one, two or three dose of the pharmaceutical composition over the entire course of period prevention is desired. In certain aspects, complete prevention can be achieved using a single dose of the pharmaceutical composition. In a particular aspect, complete prevention is achieved using a single dose of a pharmaceutical composition comprising about 1200 mg of oritavancin.

As used herein, a “subject” means an animal, such as a mammal, including humans, other higher primates, lower primates, and animals of veterinary importance, such as dogs, cats, horses, sheep, goats, and cattle and the like. The subject may have a bacterial infection, may be at risk for developing a bacterial infection, or may be at greater risk than the general population for exposure to infectious bacteria.

As used herein, the terms “dose”, “unit dose”, “dosage”, “effective dose” and related terms refer to physically discrete units that contain a predetermined quantity of active ingredient calculated to produce a desired therapeutic effect. A single dose is thus a predetermined quantity of oritavancin that is administered to a subject. Preferably, as disclosed in the methods of the invention, the oritavancin is formulated as a pharmaceutical composition for administration to the subject.

As used herein, the term “course of therapy” depends on the particular method of the invention, however the term generally means the period of time within which or over which a selected goal is achieved. In terms of methods of treatment, the course of therapy is the time period which is required to achieve treatment of the bacterial infection in the subject. In terms of methods of prophylaxis, the course of therapy is the period of time over which prophylaxis for a bacterial infection is achieved. In terms of methods of prevention, the course of therapy is the period of time over which prevention from a bacterial infection is achieved.

Although the invention is directed to the treatment or prevention of bacterial infections, the invention encompasses therapeutic and prophylactic methods against other diseases caused by or related to bacterial infection, including but not limited to otitis, conjunctivitis, pneumonia, bacteremia, sinusitis, pleural empyema and endocarditis, intravascular or endothelial infections, osteomyelitis and meningitis. In such methods, oritavancin is administered to a subject in an amount sufficient to provide a therapeutic effect and thereby prevent or treat the infection of the subject.

While oritavancin and pharmaceutical compositions can be administered in a systemic manner through the means described above, they may also be administered in a localized manner. For example, the active agents may be administered directly, such as through a topical composition or directly into a subcutaneous or other form of wound.

### Oritavancin

Oritavancin diphosphate (oritavancin; also known as N^{DISACC}-(4-(4-chlorophenyl)benzyl)A82846B and LY333328) is a semi-synthetic lipoglycopeptide derivative of a naturally occurring glycopeptide. Its structure confers potent antibacterial activity against gram-positive bacteria, including vancomycin-resistant enterococci (VRE), methicillin- and vancomycin-resistant staphylococci, and penicillin-resistant streptococci. The rapidity of its bactericidal activity against exponentially-growing S. aureus (≧3-log reduction within 15 minutes to 2 hours against MSSA, MRSA, and VRSA) is one of the features that distinguishes it from the prototypic glycopeptide vancomycin (McKay et al., J Antimicrob Chemother. 63(6):1191-9 (2009), Epub 2009 Apr. 15).

Oritavancin has the following chemical structure:

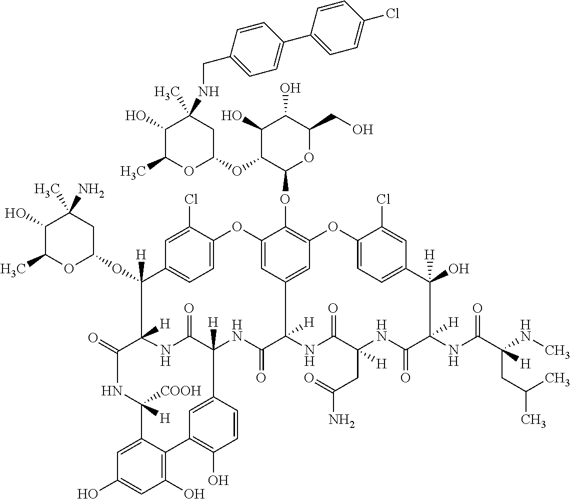 [SMILES: CN[C@H](CC(C)C)C(=O)N[C@H]1C(=O)N[C@@H](CC(N)=O)C(=O)N[C@H]2C(=O)N[C@H]3C(=O)N[C@H](C(=O)N[C@H](C(=O)O)c4cc(O)cc(O)c4-c4cc3ccc4O)[C@H](O[C@H]3C[C@](C)(N)[C@@H](O)[C@H](C)O3)c3ccc(c(Cl)c3)Oc3cc2cc(c3O[C@@H]2O[C@H](CO)[C@@H](O)[C@H](O)[C@H]2O[C@H]2C[C@](C)(NCc3ccc(-c4ccc(Cl)cc4)cc3)[C@@H](O)[C@H](C)O2)Oc2ccc(cc2Cl)[C@H]1O]

Oritavancin may be used per se in the methods of the present invention, or in the form of a pharmaceutically acceptable salt, hydrate, solvate, or mixtures thereof. The term “pharmaceutically acceptable salt” refers to non-toxic acid addition salts derived from inorganic and organic acids. While reference is made herein to both “oritavancin” and “a pharmaceutically acceptable salt thereof”, the term “oritavancin” should be understood to include both the compound per se as well as a pharmaceutically acceptable salt, hydrate, solvate, or a mixture thereof, unless otherwise indicated by context, as the term “oritavancin” alone may be used for the sake of brevity.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenylsulfonic acid, carbonic acid, succinic acid, citric acid, benzoic acid, acetic acid, and the like. Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counter-ion forming a part of any salt of this invention is not of a critical nature, so long as the salt as a whole is pharmacologically acceptable and as long as the counter-ion does not contribute undesired qualities to the salt as a whole.

Means for the preparation of the glycopeptide antibiotics, including oritavancin and analogs thereof, may be found, for example, in U.S. Pat. No. 5,840,684, incorporated herein by reference in its entirety.

### Pharmaceutical Compositions

In each of the methods of the present invention, oritavancin may be administrated to the subject in the form of a pharmaceutical composition. The pharmaceutical compositions of the invention comprise oritavancin, or a pharmaceutically acceptable salt, hydrate, solvate, or a mixture thereof, and a pharmaceutically acceptable carrier or excipient. In specific aspects, the pharmaceutical compositions of the invention comprise oritavancin, or a pharmaceutically acceptable salt thereof.

Pharmaceutically acceptable carriers and excipient are those compounds, solutions, substances or materials that can be used to produce formulations of oritavancin that are suitable for administered to a subject, such as a human. In particular, carriers and excipients of the present invention are those useful in preparing a pharmaceutical composition that is generally safe, non-toxic and neither biologically nor otherwise undesirable and that may present pharmacologically favorable profiles, and includes carriers and excipient that are acceptable for veterinary use as well as human pharmaceutical use. Suitable pharmaceutically acceptable carriers and excipients are well known in art and can be determined by those of skill in the art as the clinical situation warrants. The skilled artisan will understand that diluents are included within the scope of the terms carriers and excipients. Examples of suitable carriers and excipients include dextrose, water, glycerol, ethanol, propylene glycol, polysorbate 80 (Tween-80™), poly(ethylene)glycol 300 and 400 (PEG 300 and 400), PEGylated castor oil (e.g. Cremophor EL), poloxamer 407 and 188, a cyclodextrin or a cyclodextrin derivative (including HPCD ((2-hydroxypropyl)-cyclodextrin) and (2-hydroxyethyl)-cyclodextrin; see, e.g., U.S. patent application publication 20060194717), hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, and liposomes. The terms specifically exclude cell culture medium. More particularly: (1) 5% (w/v) dextrose, or (2) water, may be used as a pharmaceutically acceptable carrier.

Excipients included in a formulation have different purposes depending, for example on the nature of the drug, and the mode of administration. Examples of generally used excipients include, without limitation: stabilizing agents, solubilizing agents and surfactants, buffers, antioxidants and preservatives, tonicity agents, bulking agents, lubricating agents, emulsifiers, suspending or viscosity agents, inert diluents, fillers, disintegrating agents, binding agents, wetting agents, lubricating agents, antibacterials, chelating agents, sweeteners, perfuming agents, flavoring agents, coloring agents, administration aids, and combinations thereof.

The compositions may contain common carriers and excipients, such as cornstarch or gelatin, lactose, sucrose, microcrystalline cellulose, kaolin, mannitol, dicalcium phosphate, sodium chloride, alginic acid, croscarmellose sodium, and sodium starch glycolate.

The particular carrier, diluent or excipient used will depend upon the means and purpose for which the active ingredient is being applied.

Pharmaceutically acceptable excipients also include tonicity agents that make the composition compatible with blood. Tonicity agents are particularly desirable in injectable formulations.

Acceptable methods for preparing the pharmaceutical compositions according to the invention are known to those skilled in the art. For example, pharmaceutical compositions may be prepared following conventional techniques of the pharmaceutical chemist involving steps such as mixing, granulating, and compressing when necessary for tablet forms, or mixing, filling, and dissolving the ingredients as appropriate, to give the desired products for various routes of administration.

The invention is also drawn to pharmaceutical compositions for use in treating or preventing a bacterial skin infection in a subject, wherein the compositions comprise oritavancin or a pharmaceutically acceptable salt thereof. In one aspect, the compositions comprise about 1200 mg.

### Modes of Administration

The pharmaceutical compositions and compounds of the present invention may be formulated, for example, for oral, enteral, sublingual, intranasal, intraocular, rectal, intravaginal, transdermal, mucosal, topical or parenteral administration. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection or infusion of drug formulations can be used to effect such administration. In certain aspects of each of the embodiments of the invention, the pharmaceutical composition is administered to the subject intravenously.

Formulations for parenteral administration can be in the form of aqueous or non-aqueous isotonic sterile solutions, suspensions or fat emulsions. The unit dosage of these solutions or suspensions can be in a concentrated liquid, powder or granular form for ex tempore reconstitution in the appropriate pharmaceutically acceptable carrier, such as sterile water, at the time of delivery. In addition to the above-mentioned excipients, powder forms optionally include bulking agents (e.g. mannitol, glycine, lactose, sucrose, trehalose, dextran, hydroxyethyl starch, ficoll and gelatin), and cryo or lyoprotectants. In an alternative embodiment, the parenteral unit dosage form of pharmaceutical compositions and compounds of the present invention can be a ready-to-use solution of the pharmaceutical compositions and compounds in a suitable carrier in sterile, hermetically sealed ampoules or in sterile pre-loaded syringes. The suitable carrier optionally comprises any of the above-mentioned excipients. The parenteral form used for injection must be fluid to the extent that easy syringability exists.

Excipients used in parenteral preparations may also include, without limitation, stabilizing agents (e.g. carbohydrates, amino acids and polysorbates, such as 5% dextrose), solubilizing agents (e.g. cetrimide, sodium docusate, glyceryl monooleate, polyvinylpyrolidone (PVP) and polyethylene glycol (PEG)), surfactants (e.g. polysorbates, tocopherol PEG succinate, poloxamer and Cremophor™), buffers (e.g. acetates, citrates, phosphates, tartrates, lactates, succinates, amino acids and the like), antioxidants and preservatives (e.g. BHA, BHT, gentisic acids, vitamin E, ascorbic acid, sodium ascorbate and sulfur containing agents such as sulfites, bisulfites, metabisulfites, thioglycerols, thioglycolates and the like), tonicity agents (for adjusting physiological compatibility), suspending or viscosity agents, antibacterials (e.g. thimersol, benzethonium chloride, benzalkonium chloride, phenol, cresol and chlorobutanol), chelating agents, and administration aids (e.g. local anesthetics, anti-inflammatory agents, anti-clotting agents, vaso-constrictors for prolongation and agents that increase tissue permeability), and combinations thereof.

Parenteral formulations using hydrophobic carriers include, for example, fat emulsions and formulations containing lipids, lipospheres, vesicles, particles and liposomes. Fat emulsions include in addition to the above-mentioned excipients, a lipid and an aqueous phase, and additives such as emulsifiers (e.g. phospholipids, poloxamers, polysorbates, and polyoxyethylene castor oil), and osmotic agents (e.g. sodium chloride, glycerol, sorbitol, xylitol and glucose). Liposomes include natural or derived phospholipids and optionally stabilizing agents such as cholesterol.

In intravenous (IV) use, a sterile formulation of the pharmaceutical compositions of the present invention and optionally one or more additives, including solubilizers or surfactants, can be dissolved or suspended in any of the commonly used intravenous fluids and administered by infusion. Intravenous fluids include 5% dextrose in water.

In intramuscular preparations, a sterile formulation of the pharmaceutical compositions of the present invention can be dissolved and administered in a pharmaceutical diluent such as Water-for-Injection (WFI) or 5% dextrose in water. A suitable insoluble form of the pharmaceutical compositions may be prepared and administered as a suspension in an aqueous base or a pharmaceutically acceptable oil base, e.g. an ester of a long chain fatty acid such as ethyl oleate.

### IV. Examples

### Example 1

### Summary

Oritavancin is a lipoglycopeptide with rapid bactericidal activity against gram-positive bacteria including methicillin-resistant Staphylococcus aureus (MRSA). Oritavancin's concentration-dependent activity and long half-life allows for single dose administration that may provide better compliance and favourable efficacy and safety outcomes.

Adults with acute bacterial skin and skin structure infections (ABSSSI) requiring IV therapy received a single 1200 mg dose of oritavancin or vancomycin for 7 to 10 days. The primary efficacy endpoint comprised: 1) cessation of spreading or reduction in the size of the baseline lesion, 2) absence of fever, and 3) no rescue antibiotic, at 48 to 72 hours, for the modified intent to treat population.

Of 968 patients randomized in the study, 475 and 479 were included in the modified intent-to-treat population for oritavancin and vancomycin, respectively. At 48 to 72 hours, both the early clinical evaluation response rate for oritavancin (primary endpoint: 82.3%) and proportion of patients attaining≧20% reduction in lesion area from baseline for oritavancin (86.9%) were noninferior to those of vancomycin (78.9% and 82.9%, respectively). Likewise, at 7 to 14 days after end of treatment, both investigator-assessed cure with oritavancin (79.6%) and sustained cure with oritavancin (67.2%) were similar to those of vancomycin (80.0% and 69.7%, respectively). Efficacy outcomes for MRSA-infected patients and adverse event profiles overall were similar between treatment groups.

A single 1200 mg dose of oritavancin was thus as efficacious as 7 to 10 days of vancomycin in treating ABSSSI suspected or proven to be caused by gram-positive pathogens including MRSA, and was well-tolerated, with a safety profile similar to vancomycin. Oritavancin has the potential to provide a single-dose alternative to multi-dose therapies for the treatment of ABSSSI.

### Methods

Study Design and Treatment.

SOLO I was a Phase 3, international, multicenter, randomized, double-blind, comparative efficacy and safety study with single dose IV oritavancin versus IV vancomycin for 7 to 10 days in adults with ABSSSI comprising wound infection, cellulitis, or major cutaneous abscess. The study design was consistent with current guidelines from FDA (U.S. Food and Drug Administration. Guidance for Industry Acute Bacterial Skin and Skin Structure Infections: Developing Drugs for Treatment (DRAFT GUIDANCE). Available via Internet at the URL containing: fda.gov/downloads/Drugs/GuidanceComplianceRegulatoryInformation/Guidances/ucm071185.p df) and EMA (European Medicines Agency Committee for Medicinal Products for Human Use. Addendum to the note for guidance on evaluation of medicinal products indicated for treatment of bacterial infections (CHMP/EWP/558/95 REV 2) to address indication-specific clinical data (draft). 2012. Available via Internet at the URL containing: ema.europa.eu/docs/en_GB/document_library/Scientific_guideline/2012/07/WC500129443.pdf) and Foundation for National Institutes of Health guidance was considered for analyses (Talbot G H etl. Clin Infect Dis. 2012. 55(8):1114-1121). The protocol was approved by institutional review boards/ethics committees and all patients provided written informed consent. The study was conducted from January 2011 through October 2012 at 46 study centers in Mexico, Spain, Germany, Romania, Russia, Ukraine, Israel, India, and the United States. Study participants were randomized in a 1:1 ratio to receive either a single 1200 mg IV dose of oritavancin followed by IV placebo or IV vancomycin (1 g or 15 mg/kg, every 12 hours) for 7 to 10 days. Aztreonam and metronidazole were permitted for gram-negative and anaerobic cover, respectively.

Randomization was stratified by geographic region, site, and presence of diabetes mellitus. An enrollment cap of 30% was maintained for major cutaneous abscesses.

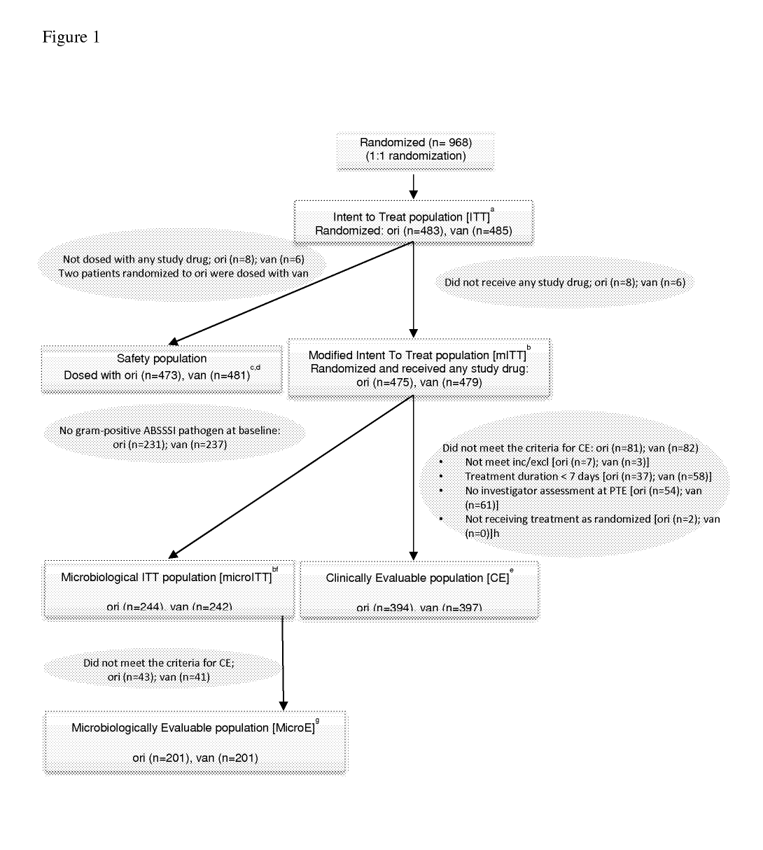

Clinical evaluations were performed at the following time points: Early Clinical Evaluation (ECE) at 48 to 72 hours after the initiation of the study drug infusion, End of Therapy (EOT) at Day 7 to Day 10 or the day the patient stopped study drug or changed to non-study drug therapy for primary ABSSSI, Day 10 Evaluation defined as 10 days after the initiation of the study drug infusion, Post Therapy Evaluation (PTE) at 7 to 14 days after the EOT visit, and Safety Follow-up at 60 days (+7 days) after the initiation of the study drug infusion. Safety data was reviewed by a data monitoring committee. The definitions of the analysis populations are provided in FIG. 1.

Patient Eligibility.

Eligible patients were to be at least 18 years of age with diagnosis of ABSSSI that was suspected or proven to be due to a gram-positive pathogen and which required at least 7 days of IV therapy. The diagnosis of ABSSSI was defined as wound infections (either traumatic or surgical in origin), cellulitis/erysipelas or major cutaneous abscess with surrounding erythema, edema, and/or induration of at least 75 cm². The onset of wound infections or cellulitis/erysipelas must have occurred within 7 days prior to randomization. Patients also had to present with signs and symptoms of systemic inflammation. Patients were not eligible to participate if they received systemic or topical antibacterial therapy with gram-positive activity within the preceding 14 days.

Efficacy Assessments.

The primary efficacy endpoint was a composite outcome of 1) cessation of spreading or reduction in the size of the baseline lesion, 2) absence of fever, and 3) no rescue antibiotic medication at the ECE visit, for the mITT population.

The secondary efficacy outcomes were as follows: 1) Lesion area decrease by ≧20% from Baseline at ECE, 2) Investigator-Assessed Clinical Response at PTE, 3) Sustained Clinical Response at PTE.

Lesion Area Decrease by ≧20% from Baseline at ECE:

The analyses were performed for patients who had lesion area assessments at baseline and ECE using the mITT and CE populations. If a patient had a missing assessment at baseline or ECE, the patient was treated as a failure

Investigator-Assessed Clinical Response at PTE:

If a patient had clinical failure assessed beyond the PTE visit window, the patient was considered as a clinical failure. If a patient had clinical success assessed beyond the PTE visit window, a) and if the patient had clinical failure before the PTE visit, the patient was considered a clinical failure at PTE or b) if the patient had clinical success also before the PTE visit, the patient was considered a clinical success at PTE. After the above imputation, patients still with missing assessment at PTE will be treated as failure

Sustained Clinical Response at PTE:

a patient was categorized as having had a clinical cure if the patient experienced a complete or nearly complete resolution of baseline signs and symptoms of the primary infection such that no further treatment with antibiotics was needed at end-of-treatment and sustained/continued resolution of infection at PTE for the mITT population. A patient was classified as a clinical failure at PTE if there was any of the following:
Did not fulfill criteria for clinical cure (defined above)
Investigator assigned failure any time prior to PTE
Patient died (all-cause mortality) following the start of study drug
Incision and drainage after 48 hours of treatment that was unplanned prior to randomization, with the exception of cellulitis where there was a conversion into an abscess or when an extension of the original incision was indicated
Persistence or worsening of erythema/induration and/or purulent drainage
Initiation of non-study, antibacterial drugs for treatment of ABSSSI
Initiation of non-study, antibacterial drugs for treatment of other infections, unless antibiotics lack efficacy in the treatment of ABSSSI
Otherwise did not meet the definition of clinical cure (eg, lost to follow-up, study drug discontinued because of an adverse reaction)
   Patients who were not classified as clinical cures were classified as failures.

Safety Assessments.

Safety assessments (Safety Population) included vital signs, electrocardiogram (ECG), clinical chemistry and hematology parameters, reporting of adverse events (AEs) and serious adverse events (SAEs). Treatment-emergent adverse events (TEAEs) were defined as AEs with onset or worsening severity at or after the first dose of study drug through the Safety Follow-up Visit (Day 60+7 days).

Statistical Methods.

A sample size of 960 patients (480 per treatment group) provided at least 90% power to test non-inferiority (NI) of oritavancin against vancomycin with respect to the primary efficacy outcome rate using 10% NI margin at the 1-sided alpha level of 0.025, when the primary efficacy outcome rate is assumed to be 75% in both treatment groups.

For the primary efficacy assessment, a 1-sided 97.5% confidence interval (CI) for the difference in rates of primary efficacy outcome between the two treatment groups (oritavancin rate minus vancomycin rate) was derived, using a two-group large-sample normal approximation test of proportions. If the lower bound of the 1-sided 97.5% CI was above −10%, non-inferiority of oritavancin was claimed at the 1-sided alpha level of 0.025.

The confirmative analyses of primary efficacy outcome were performed in the modified intent-to-treated (mITT) population. Analyses of secondary outcomes were performed in a similar manner by providing confidence intervals. Any missing assessments were considered as failures for the primary and secondary efficacy outcomes.

For safety assessments, descriptive analyses were performed in the safety population for all safety parameters by treatment group.

Analysis Population Definitions.

The intent-to-treat (ITT) population included all patients randomized into the study. The modified intent-to-treat (mITT) was the primary population for all the efficacy analyses and included all randomized patients who received any study drug. The clinically evaluable (CE) population consisted of all mITT patients who met the inclusion/exclusion criteria, received the full-course of randomized study treatment (for a minimum of 7 days), and had investigator assessment for clinical cure at PTE. The CE population was used to confirm the efficacy analyses. The microbiologically ITT (MicroITT) population consisted of all mITT patients with baseline gram-positive pathogen(s) known to cause ABSSSI and it was used for the secondary efficacy analyses. The microbiologically evaluable (MicroE) population was used to confirm the secondary efficacy analyses and consisted of all patients who were in both the MicroITT and CE populations. The safety population was the primary population for all the safety analyses, and consisted of all patients who were dosed with study drug, irrespective of randomization. Treatment classification was based on the actual treatment received.

Results

Patient Disposition and Analysis Populations.

FIG. 1 depicts the patient disposition and analysis populations in the study.

Patient Demographics and Baseline Medical Characteristics.

For the mITT population, both the oritavancin and vancomycin treatment groups had similar demographics, type of ABSSSI, and relevant medical or surgical history. The mean age of patients was 46.2 years and 44.3 years with 9.9% and 7.9% at least 65 years of age, respectively. Patients were predominantly white and male. Infection types were balanced in the oritavancin and vancomycin groups, with approximately 50% cellulitis, 30% abscess, and 20% wound infection overall. The median infection area at Baseline was 248.0 cm² for the oritavancin group and 225.6 cm² for the vancomycin group. A pathogen was isolated from approximately 60% of patients in both treatment groups at Baseline; approximately 96% of these patients had a gram-positive pathogen known to cause ABSSSI. S. aureus was the most common pathogen and MRSA was recovered from the primary ABSSSI site in 104 patients treated with oritavancin and in 100 patients treated with vancomycin.

Clinical Outcomes.

All protocol-specified primary and secondary efficacy endpoints of the SOLO 1 study met the 10% non-inferiority margin. The single 1200 mg IV dose of oritavancin demonstrated similar efficacy to 7 to 10 days of IV vancomycin at the ECE and PTE endpoints. Results in the mITT population demonstrated the following (Tables 1 and 2): (1) the response rates for oritavancin and vancomycin for the primary endpoint at ECE were comparable (82.3% vs 78.9% respectively; 3.4% difference; 95% CI −1.6 to 8.4); (2) 86.9% of patients treated with oritavancin achieved at least a 20% reduction in lesion size at ECE compared with 82.9% of patients treated with vancomycin (4.1% difference; 95% CI −0.5 to 8.6); (3) the investigator-assessed clinical response rates at 7 to 14 days after EOT (i.e., at PTE) were similar for oritavancin and vancomycin (79.6% vs 80.0%, respectively; −0.4% difference; 95% CI −5.5 to 4.7); (4) the sustained clinical response rate at PTE observed with oritavancin was comparable to vancomycin (67.2% vs 69.7%; −2.6% difference; 95% CI −8.5 to 3.3).

The number and reasons for failure at both ECE and PTE were balanced across the two treatment groups (Table S2 and Table S3). The majority of patients classified as failure at PTE were due to missing data. These results were confirmed in the CE population (Tables 1 and 2).

Oritavancin has the potential to provide a single-dose alternative to multi-dose therapies for the treatment of ABSSSI caused by MRSA since comparable efficacy was demonstrated across the two treatment groups in the MRSA subpopulation for the primary and secondary endpoints (Tables 1 and 2). Within the MRSA subpopulation of the microITT population, a numerically higher proportion of patients treated with oritavancin demonstrated at least a 20% reduction in lesion size by 48 to 72 hours compared with patients treated with vancomycin (90.4% vs 84.0%; 6.4% difference; 95% CI −2.8 to 15.5). These efficacy results for MRSA-infected patients were confirmed in the MicroE population.

TABLE 1
Primary Efficacy Endpoint at Early Clinical Evaluation (48 to 72 Hours)

| | Oritavancin % | | Vancomycin % | | Difference (95% | |
|---|---|---|---|---|---|---|
| Population | (proportion) | | (proportion) | | Confidence Interval) | |
| Modified intent-to-treat (mITT) | 82.3% | (391/475) | 78.9% | (378/479) | 3.4 | (−1.6, 8.4) |
| Clinically evaluable (CE) | 87.3% | (344/394) | 86.1% | (342/397) | 1.2 | (−3.6, 5.9) |
| MRSA (Microbiologically intent-to- | 80.8% | (84/104) | 80.0% | (80/100) | 0.8 | (−10.1, 11.7) |
| treat [MicroITT]) | | | | | | |
| MRSA (Microbiologically evaluable | 88.4% | (76/86) | 86.6% | (71/82) | 1.8 | (−8.2, 11.8) |
| [MicroE]) | | | | | | |

NOTE.
MRSA—methicillin-resistant Staphylococcus aureus.
Primary efficacy endpoint is the response rate of the composite of cessation of spread, absence of fever, and no rescue antibiotics at the early clinical evaluation (48 to 72 hours after end of treatment).

TABLE 2
Secondary Efficacy Outcomes

| | | | |
|---|---|---|---|
| | Oritavancin % | Vancomycin % | Difference (95% |
| Population | (proportion) | (proportion) | Confidence Interval) |

Lesion Size Reduction ≧20% From Baseline at the Early Clinical Evaluation (48 to 72 Hours)

| | | | | | | |
|---|---|---|---|---|---|---|
| Modified intent-to-treat (mITT) | 86.9% | (413/475) | 82.9% | (397/479) | 4.1 | (−0.5, 8.6) |
| Clinically evaluable (CE) | 90.6% | (357/394) | 88.7% | (352/397) | 1.9 | (−2.3, 6.2) |
| MRSA (Microbiologically intent-to- | 90.4% | (94/104) | 84.0% | (84/100) | 6.4 | (−2.8, 15.5) |
| treat [MicroITT]) | | | | | | |
| MRSA (Microbiologically evaluable | 96.5% | (83/86) | 89.0% | (73/82) | 7.5 | (−0.3, 15.3) |
| [MicroE]) | | | | | | |

Investigator-Assessed Clinical Cure at the Post Therapy Evaluation (7 to 14 Days)

| | | | | | | |
|---|---|---|---|---|---|---|
| Modified intent-to-treat (mITT) | 79.6% | (378/475) | 80.0% | (383/479) | −0.4 | (−5.5, 4.7) |
| Clinically evaluable (CE) | 91.9% | (362/394) | 93.2% | (370/397) | −1.3 | (−5.0, 2.3) |
| MRSA (Microbiologically intent-to- | 82.7% | (86/104) | 83.0% | (83/100) | −0.3 | (−10.7, 10.0) |
| treat [MicroITT]) | | | | | | |
| MRSA (Microbiologically evaluable | 95.3% | (82/86) | 97.6% | (80/82) | −2.2 | (−7.8, 3.4) |
| [MicroE]) | | | | | | |

Sustained Clinical Cure at the Post Therapy Evaluation (7 to 14 Days)

| | | | | | | |
|---|---|---|---|---|---|---|
| Modified intent-to-treat (mITT) | 67.2% | (319/475) | 69.7% | (334/479) | −2.6 | (−8.5, 3.3) |
| Clinically evaluable (CE) | 79.4% | (313/394) | 83.4% | (331/397) | −3.9 | (−9.3, 1.5) |
| MRSA (Microbiologically intent-to- | 68.3% | (71/104) | 69.0% | (69/100) | −0.7 | (−13.5, 12.0) |
| treat [MicroITT]) | | | | | | |
| MRSA (Microbiologically evaluable | 81.4% | (70/86) | 84.1% | (69/82) | −2.8 | (−14.2, 8.7) |
| [MicroE]) | | | | | | |

NOTE:
MRSA—methicillin-resistant Staphylococcus aureus.

Safety and Tolerability.

The incidence of TEAEs, regardless of relationship to study drug, was similar between oritavancin and vancomycin groups; TEAEs were primarily mild in severity. The most frequently reported AEs in the oritavancin group were nausea (11.0% versus 8.9% in the vancomycin group), headache (7.2% versus 7.9%), vomiting (4.9% versus 3.7%), and diarrhea (4.9% versus 3.5%). Of the most frequently reported TEAEs, the incidence of pruritus and infusion site reactions was lower in the oritavancin group compared with the vancomycin group. No histamine-like reactions occurred in the oritavancin group compared to 2 patients in the vancomycin group who experienced Red Man syndrome. The proportion of patients experiencing a TEAE that led to discontinuation of the study drug was lower in the oritavancin group (3.8%) than in the vancomycin group (5.8%). In the oritavancin treatment group, the only TEAE that led to discontinuation in more than one patient was cellulitis which was reported in 2 patients. In the vancomycin group, TEAEs that led to discontinuation of study drug in more than one patient were hypersensitivity (5 patients), cellulitis (3 patients), and sepsis, skin bacterial infection, drug hypersensitivity, pruritus, and rash (each with 2 patients).

The frequency and distribution of SAEs was similar in both groups (7.4% versus 7.3% respectively). Cellulitis was the most frequently reported SAE in both treatment groups (5 patients [1.1%] with oritavancin versus 8 patients [1.7%] with vancomycin) of which 2 and 4 of these events, respectively were considered new events, and of these patients, only 2 and 3 patients, respectively, discontinued the study drug due to cellulitis. In addition, the vancomycin group had 2 patients that had a SAE of skin bacterial infection that led to discontinuation of study drug. Three patients died during the study: one patient treated with oritavancin died due to sepsis/septic shock and two patients treated with vancomycin died, one from sepsis and the other from advanced dementia with Parkinsonism, all of which were determined by the investigator to be unrelated to the study drug.

The incidence of laboratory abnormalities, including liver function tests, was similar between the treatment groups. No difference of vital signs and ECG findings was identified between the treatment groups.

### Discussion

The study demonstrated that a single 1200 mg dose of oritavancin administered to adults was safe and as efficacious as 7 to 10 days of vancomycin for the treatment of ABSSSI caused by gram-positive pathogens including MRSA, and has the potential to provide a single-dose alternative for the treatment of ABSSSI. Currently available therapeutic options for the treatment of ABSSSI require repeat IV administrations which may result in extended hospitalization and can incur significant costs to the health care system. A single-dose treatment for ABSSSI that achieves early and sustained clinical response could potentially reduce complications associated with multiple IV administrations, reduce the need for health care resources, and improve compliance.

Oritavancin met all primary and secondary endpoints. Oritavancin was shown to be non-inferior to vancomycin in the efficacy analyses for the ECE (48 to 72 hour) endpoints required by the U.S. Food and Drug Administration (FDA) and the later (7-14 days after end of treatment) endpoint required by the European Medicines Agency (EMA). Efficacy demonstrated at ECE with oritavancin (lesion size reduction of ≧20%) was sustained since more than 84% of patients were both early treatment responders and clinical successes at PTE in the CE population. Oritavancin demonstrated efficacy against isolates commonly linked to ABSSSIs, including S. aureus. and of special interest, MRSA. The efficacy was similar in the overall population and in those patients with microbiologically-confirmed MRSA.

The single-dose oritavancin administration regimen may provide better clinical and economic outcomes, such as reduction in nosocomial infections, ensured patient compliance, reduced length of hospital stay or elimination of in-patient hospital stays, reduced ambulatory care resources, and reduced health care utilization. The efficacy of a single dose of oritavancin in ABSSSIs is similar to that obtained by other antibiotics, but when given in multiple doses: tedizolid: 6 days, ceftaroline: 5 to 14 days, daptomycin: 7 to 14 days, dalbavancin: Days 1 and 8, tigecycline: up to 14 days, telavancin: 7 to 14 days, ceftobiprole: 9 days, linezolid: 10 to 14 days, vancomycin: up to 14 days.

A single dose of oritavancin was demonstrated to be safe and well-tolerated. The frequency, distribution, and severity of AEs were similar for oritavancin and vancomycin. Discontinuations due to AEs were uncommon, and there were no deaths assessed as related to study drug in either treatment group. Nausea and headache were the most commonly reported TEAEs for oritavancin versus pruritus and nausea for vancomycin. Of note, post-hoc analysis of AEs showed that oritavancin-treated patients suffered fewer pruritus (3.4% vs. 9.1%; p value<0.001) and infusion site reaction AEs (4.0% vs. 7.1%; p value=0.040). There were fewer skin and subcutaneous tissue adverse events (11.6% vs. 19.1%; p value=0.001) among patients treated with oritavancin. There were no clinically significant differences between oritavancin and vancomycin for any hematologic or clinical laboratory parameters. Oritavancin's long half-life (Rubino et al 2009) was not associated with any untoward safety effects as assessed throughout the study including the Day 60 follow up. The number of patients with opportunistic infections, sepsis, and septic shock was negligible in both groups.

### Example 2

### Summary

Serious infections caused by methicillin-resistant Staphylococcus aureus (MRSA) have been documented both in healthcare and community settings, and continue to be problematic in healthcare systems. Traditionally, MRSA isolates are positively identified by amplification of a portion of the mecA gene (CLSI, 2012a). Recently, isolates which are phenotypically resistant to methicillin but which are mecA amplification-negative have been described (Garcia-Alvarez et al., 2011). Further analysis of some of these strains indicated that they harbour a novel mec gene which is only ˜70% homologous to the mecA gene. This gene has been designated the mecC gene (Ito et al., 2012) and has been shown to be present in zoonotic MRSA isolates (Petersen et al., 2012). This study evaluated the activity of oritavancin and comparators against 14 mecC-carrying MRSA strains obtained from skin and skin structure infections by broth microdilution MIC and time-kill assays at clinically-relevant concentrations of drugs. Two MRSA strains carrying the mecA gene were also included in the study.

### Methods

Strains:

The S. aureus (SA) strains used in this study were: ATCC 43300 (mecA methicillin-resistant SA [MRSA]); ATCC 33591 (mecA MRSA), ATCC 29213 (methicillin-susceptible SA; quality control strain) and 14 mecC MRSA obtained from the Staten Serum Institute (Copenhagen, Denmark).

Minimal Inhibitory Concentration (MIC) Determination:

MICs of oritavancin (ORI), vancomycin (VAN), daptomycin (DAP) and linezolid (LZD) were determined by broth microdilution assay according to M7-A9 and M100-S22 guidelines (CLSI, 2012 a & b). Tests with oritavancin included polysorbate-80 (final test concentration of 0.002%) as described (CLSI 2012a; Arhin et al., 2008). Tests with daptomycin included CaCl₂ at a concentration of 50 mg/L. MICs were read after 24 h incubation.

Time-Kill Kinetics:

Time-kill assays followed guideline M26-A of the CLSI (NCCLS, 1999) with the following changes. Exponential-phase cells grown in cation-adjusted Mueller-Hinton broth (CAMHB) were diluted to approximately 5×10⁵ colony forming units (CFU)/mL) and exposed to ORI and comparator antibiotics over 24 h in CAMHB in a 96-well deep plate. For assays with ORI, CAMHB was supplemented with 0.002% polysorbate-80 (Arhin et al., 2008; CLSI, 2012a); for assays with DAP, CAMHB was supplemented with 50 mg/L CaCl₂ (final concentration; CLSI, 2012b). Viable cell count was determined by serial dilution plating, including the use of 25 g/L charcoal suspension to limit antibiotic carryover. Bactericidal activity was defined as a ≧3 log decrease in cell counts at 24 h, relative to initial inoculum (NCCLS, 1999). Time-kill assays were repeated at least twice independently; results presented are from a representative experiment.

Antibiotic Concentration Selection:

ORI concentrations were chosen to approximate free peak (fCₘₐₓ; defined as the peak concentration of non-protein bound drug) and free trough (fCₘᵢₙ; defined for ORI as the concentration of non-protein bound drug at 24 h) levels in plasma following administration of a 1200 mg dose (Belley et al., 2013 Table 10). ORI protein binding is estimated at approximately 85% in serum from nonclinical species and humans (Arhin et al., 2010). Doubling dilution concentrations of comparator agents were likewise chosen to approximate their fCₘₐₓ and fCₘᵢₙ levels in plasma when administered at approved dosages for complicated skin and skin structure infections, using pharmacokinetic data and protein binding values from their respective package inserts (DAP [Cubicin®]; LZD [Zyvox®]; VAN [Vancocin®]; Table 3).

TABLE 3

| | | | | Doubling Dilution Concentration | |
|---|---|---|---|---|---|
| | Free | Representative | | (mg/L) Representing | |
| Agent | Fraction | human dose | | Predicted fCₘₐₓ | Predicted fCₘᵢₙ |
| Oritavancin | 0.15 | 1200 | mg | 16 | 2 |
| Vancomycin | 0.54 | 1000 | mg | 16 | 4 |
| Daptomycin | 0.08 | 4 | mg/kg | 4 | 0.5 |
| Linezolid | 0.69 | 500 | mg | 8 | 2 |

### Results

The oritavancin MIC against the mecC strains ranged from 0.03-0.06 mg/L and was within a doubling dilution of oritavancin MIC for the QC and mec A strains (0.06 mg/L; Table 4). The vancomycin MIC (1 mg/L) was identical for all strains tested. Daptomycin and linezolid MICs were within one doubling dilution (0.5-1 mg/L and 1-2 mg/L), respectively, for all strains tested. MIC₉₀ values for the mecC strains were 0.06, 1, 1 and 2 mg/L for oritavancin, vancomycin, daptomycin and linezolid, respectively.

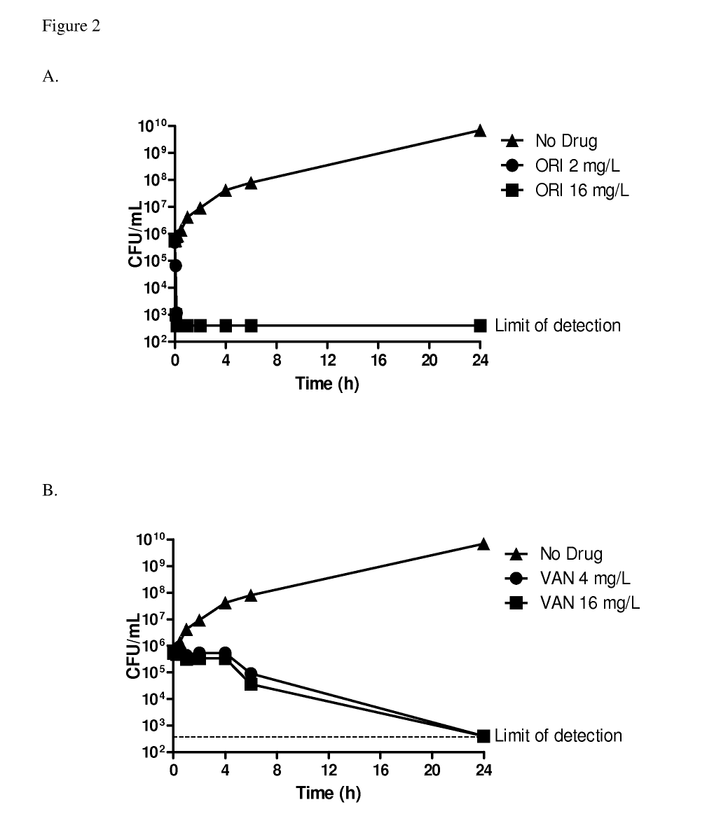

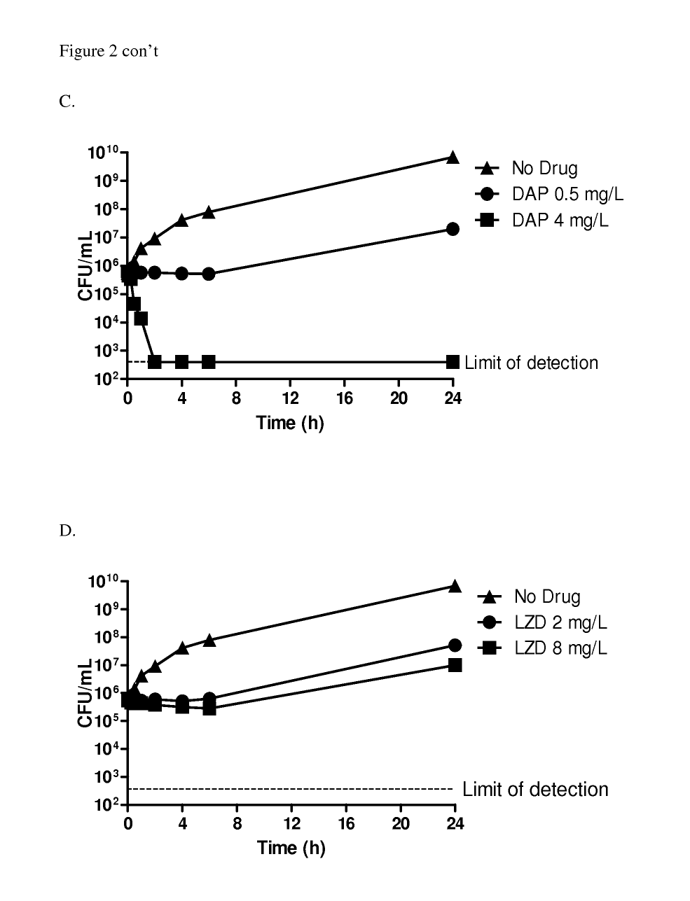

Table 5 summarizes results from in vitro time-kill assays at pharmacologically-relevant concentrations of oritavancin and comparators against the S. aureus strains. Oritavancin at both fCₘᵢₙ and fCₘₐₓ was profoundly bactericidal, reducing inoculum by at least 3 log (99.9% kill) within ≦5 min to 15 min for all tested strains, regardless of phenotype. Vancomycin was likewise bactericidal at both fCₘᵢₙ and fCₘₐₓ, achieving 3 log kill at between 6 to 24 hours against all tested strains. Daptomycin at its fCₘᵢₙ had little to no effect or was bacteriostatic. Daptomycin at its fCₘₐₓ was bactericidal against all strains, achieving 3 log kill at between 30 min and 2 hours. Linezolid at both fCₘᵢₙ and fCₘₐₓ had little to no effect or was bacteriostatic against all tested strains. FIG. 2 shows kill curves of the tested drugs for a typical mecC strain (70611).

TABLE 4
Oritavancin and comparator MICs (mg/L)
for S. aureus isolates tested

| Strain | Phenotype | ORI | VAN | DAP | LZD |
|---|---|---|---|---|---|
| ATCC 29213 | MSSA | 0.06 | 1 | 0.5 | 2 |
| ATCC 33591 | MRSA (mecA) | 0.06 | 1 | 1 | 2 |
| ATCC 43300 | MRSA (mecA) | 0.06 | 1 | 0.5 | 1 |
| 73617 | MRSA (mecC) | 0.03 | 1 | 1 | 2 |
| 75814 | MRSA (mecC) | 0.06 | 1 | 0.5 | 2 |
| 57147 | MRSA (mecC) | 0.03 | 1 | 0.5 | 2 |
| 73829 | MRSA (mecC) | 0.03 | 1 | 0.5 | 2 |
| 78651 | MRSA (mecC) | 0.06 | 1 | 0.5 | 2 |
| 77950 | MRSA (mecC) | 0.03 | 1 | 0.5 | 2 |
| 77431 | MRSA (mecC) | 0.03 | 1 | 1 | 2 |
| 76918 | MRSA (mecC) | 0.03 | 1 | 0.5 | 2 |
| 70611 | MRSA (mecC) | 0.06 | 1 | 0.5 | 2 |
| 73415 | MRSA (mecC) | 0.06 | 1 | 1 | 2 |
| 73348 | MRSA (mecC) | 0.06 | 1 | 0.5 | 2 |
| 77964 | MRSA (mecC) | 0.06 | 1 | 0.5 | 2 |
| 78085 | MRSA (mecC) | 0.06 | 1 | 1 | 2 |
| 77601 | MRSA (mecC) | 0.03 | 1 | 0.5 | 2 |

*ATCC, American Type Culture Collection; MSSA, methicillin-susceptible Staphylococcus aureus; MRSA, methicillin-resistant Staphylococcus aureus; ORI, oritavancin; VAN, vancomycin.

TABLE 5
Activity of oritavancin and comparator agents against S. aureus strains in time-kill
assays^{a}

| | Oritavancin | | | Vancomycin | | Daptomycin | | Linezolid | |
|---|---|---|---|---|---|---|---|---|---|
| Strain (genotype) | ƒCₘᵢₙ^{c} | ƒCₘₐₓ^{c} | | ƒCₘᵢₙ | ƒCₘₐₓ | ƒCₘᵢₙ | ƒCₘₐₓ | ƒCₘᵢₙ | Cₘₐₓ |
| ATCC 29213^{b} | 10-15 min | 5-10 | min | 6-24 h | 6-24 h | +3.9 | 2-4 h | +2.2 | −2.8 |
| ATCC 33591 | 10-15 min | 5-10 | min | 6-24 h | 6-24 h | +3.8 | 30 min-1 h | +2.4 | −2.0 |
| (mecA) | | | | | | | | | |
| ATCC 43300 | 5-10 min | 5-10 | min | 6-24 h | 6-24 h | +3.6 | 1-2 h | +2.6 | −2.6 |
| (mecA) | | | | | | | | | |
| 73617 (mecC) | 10-15 min | 5-10 | min | 6-24 h | 6-24 h | +3.4 | 1-2 h | +3.4 | −1.8 |
| 75814 (mecC) | 5-10 min | ≦5 | min | 6-24 h | 6-24 h | +3.6 | 1-2 h | +2.2 | −1.3 |
| 57147 (mecC) | 5-10 min | 5-10 | min | 6-24 h | 6-24 h | +2.7 | 1-2 h | +1.6 | −2.3 |
| 73829 (mecC) | 5-10 min | ≦5 | min | 6-24 h | 6-24 h | +2.1 | 1-2 h | +1.2 | −1.8 |
| 78651 (mecC) | 10-15 min | 5-10 | min | 6-24 h | 6-24 h | +1.6 | 30 min-1 h | +1.7 | −1.4 |
| 77950 (mecC) | 5-10 min | ≦5 | min | 6-24 h | 6-24 h | +0.9 | 1-2 h | +0.9 | −1.2 |
| 77431 (mecC) | 5-10 min | ≦5 | min | 6-24 h | 6-24 h | +1.9 | 1-2 h | +2.3 | −0.5 |
| 76918 (mecC) | 5-10 min | 5-10 | min | 6-24 h | 6-24 h | +1.5 | 1-2 h | +1.8 | −0.5 |
| 70611 (mecC) | 5-10 min | ≦5 | min | 6-24 h | 6-24 h | +1.5 | 1-2 h | +1.8 | +1.5 |
| 73415 (mecC) | 5-10 min | 5-10 | min | 6-24 h | 6-24 h | +1.5 | 2-4 h | +1.2 | +1.5 |
| 73348 (mecC) | 5-10 min | 5-10 | min | 6-24 h | 6-24 h | +2.6 | 30 min-1 h | +2.5 | +1.4 |
| 77964 (mecC) | 5-10 min | ≦5 | min | 6-24 h | 6-24 h | +1.2 | 1-2 h | +1.2 | +0.2 |
| 78085 (mecC) | 10-15 min | 5-10 | min | 6-24 h | 6-24 h | +1.5 | 2-4 h | +1.5 | −0.2 |
| 77601 (mecC) | 5-10 min | ≦5 | min | 6-24 h | 6-24 h | +0.1 | 30 min-1 h | +0.5 | −1.2 |

^{a}Time to cidality is indicated for drugs that achieved bactericidal activity (≧ 3 log decrease in cell density relative to initial inoculum). Change in cell density (in log CFU/mL) at 24 h relative to starting inoculum is indicated for drugs with bacteriostatic activity (0 to < 3 log kill at 24 h relative to initial inoculum) and for drugs with little to no inhibition of growth relative to growth control at 24 h.
^{b}ATCC 29213 is a methicillin-susceptible S. aureus (MSSA) QC strain
^{c}ƒCₘₐₓ and ƒCₘᵢₙ, peak and trough concentration, respectively, of non-protein bound drug at approved doses for acute bacterial skin and skin structure infections. For oritavancin, the values are as predicted in patients receiving a single 1200 mg dose. Since ƒCₘᵢₙ is irrelevant for the single oritavancin dose, an estimate of the free oritavancin concentration at 24 h from this 1200 mg dose was used in place of the ƒCₘᵢₙ.

### Conclusions

Oritavancin in vitro activity against mecC MRSA strains was indistinguishable from that against mecA MRSA strains in both MIC and time-kill assays. Comparator MICs against the tested strains were identical or within a doubling dilution, regardless of strain phenotype. The oritavancin MIC₉₀ of the mecC MRSA strains (0.06 mg/L) was identical to the MICs of the mecA strains tested in this study and within a doubling dilution of the MIC₉₀ (0.12 mg/L) of MRSA isolates in a surveillance study (Arhin et al., 2009). Oritavancin at pharmacologically-relevant concentrations was bactericidal in time-kill assays against all the tested strains, reaching cidality between ≦5 minutes and 15 minutes. Based on MIC₉₀ and time-to-bactericidal activity values, oritavancin was more potent in vitro than vancomycin, daptomycin and linezolid against the tested mecC MRSA strains.

### Example 3

A clinical trial (SOLO II) was found to meet all protocol-specified endpoints comparing one single intravenous dose of oritavancin to twice-daily vancomycin intravenous dosing for 7-10 days. Pooled data from two clinical trials (SOLO I and SOLO II) in MRSA showed a higher proportion of oritavancin patients achieved the endpoint of >=20% reduction of lesion area at 48-72 hours.

Complete results were obtained from a phase 3 clinical trial program of oritavancin (SOLO), which investigated the drug for the treatment of acute bacterial skin and skin structure infections (ABSSSI) caused by susceptible gram-positive bacteria, including methicillin-resistant Staphylococcus aureus (MRSA). SOLO I and SOLO II clinical trials were identical multicenter, double-blind, randomized clinical trials.

In SOLO II, all protocol-specified primary and secondary efficacy endpoints were met. Oritavancin was shown to be non-inferior to vancomycin in the efficacy analyses for the Early Clinical Evaluation (ECE) (or 48-72 hour) endpoints required by the U.S. Food and Drug Administration (FDA) and the later Post Therapy Evaluation (PTE) (7-14 days after end of treatment) endpoint required by the European Medicines Agency (EMA). The efficacy was similar in the overall population and in those patients with microbiologically confirmed MRSA infections.

In findings of the combined SOLO trials, oritavancin was shown to be non-inferior to vancomycin in the efficacy analyses for the ECE and PTE endpoints. In microbiologically confirmed MRSA patients, efficacy was similar in both treatment groups; however, a higher proportion of MRSA-infected patients in the oritavancin group achieved the endpoint of greater than or equal to 20% reduction of lesion area at ECE (p value=0.032). Under the Special Protocol Assessment (SPA) agreed with the FDA, the protocols pre-specified that MRSA patients from the SOLO studies would be pooled for the evaluation of efficacy.

The combined SOLO studies represented 1,987 patients in an intent-to-treat population for evaluation of oritavancin in the treatment of ABSSSI in controlled clinical trials and assessed one of the largest subsets of patients with documented MRSA infection (405 patients).

An important finding of the SOLO studies was in the pre-specified pooled where more oritavancin-treated MRSA patients had a greater than or equal to 20% reduction in their skin lesion at just three days after treatment initiation.

TABLE 6
Primary Results in SOLO II (mITT population)

| | | Oritavancin | Vancomycin | % Difference |
|---|---|---|---|---|
| Timepoint | Endpoint | (n = 503) | (n = 502) | (95% CI) |
| Early clinical | FDA primary endpoint: | 403 (80.1%) | 416 (82.9%) | −2.7 (−7.5, 2.0) |
| evaluation (ECE) | Cessation of spread, | | | |
| | absence of fever, no | | | |
| | rescue antibiotics | | | |
| | ≧20% reduction of | 432 (85.9%) | 428 (85.3%) | 0.6 (−3.7, 5.0) |
| | lesion area | | | |
| Post therapy | EMA primary endpoint: | 416 (82.7%) | 404 (80.5%) | 2.2 (−2.6, 7.0) |
| evaluation (PTE) | Investigator-assessed | | | |
| | clinical cure | | | |

TABLE 7
Results in patients with confirmed MRSA infections
in SOLO II (Microbiological ITT population)

| | | Oritavancin | Vancomycin |
|---|---|---|---|
| Timepoint | Endpoint | (n = 100) | (n = 101) |
| Early clinical | FDA primary endpoint: | 82 (82.0%) | 82 (81.2%) |
| evaluation (ECE) | Cessation of spread, | | |
| | absence of fever, no | | |
| | rescue antibiotics | | |
| | ≧20% reduction of | 96 (96.0%) | 91 (90.1%) |
| | lesion area | | |
| Post therapy | EMA primary endpoint: | 84 (84.0%) | 86 (85.1%) |
| evaluation (PTE) | Investigator-assessed | | |
| | clinical cure | | |

Safety profiles, measured at any point up to 60 days after treatment, were similar across treatment groups in SOLO II. Overall, 50.9% of patients on oritavancin and 50.2% of patients on vancomycin were reported to experience at least one adverse event. In SOLO II treatment emergent adverse events considered by investigators as related to study drug were reported by similar proportions of patients treated with oritavancin and vancomycin (21.7% and 25.5%, respectively).

TABLE 8
Primary Results in SOLO I and SOLO II Combined (mITT population)

| | | Oritavancin | Vancomycin | % Difference |
|---|---|---|---|---|
| Timepoint | Endpoint | (n = 978) | (n = 981) | (95% CI) |
| Early clinical | FDA primary endpoint: | 794 (81.2%) | 794 (80.9%) | 0.2 (−3.3, 3.7) |
| evaluation (ECE) | Cessation of spread, | | | |
| | absence of fever, no | | | |
| | rescue antibiotics | | | |
| | ≧20% reduction of | 845 (86.4%) | 825 (84.1%) | 2.3 (−0.9, 5.4) |
| | lesion area | | | |
| Post therapy | EMA primary endpoint: | 794 (81.2%) | 787 (80.2%) | 1.0 (−2.5, 4.5) |
| evaluation (PTE) | Investigator-assessed | | | |
| | clinical cure | | | |

TABLE 9
Results in patients with confirmed MRSA infections in
SOLO I and SOLO II Combined (MicroITT population)

| | | Oritavancin | Vancomycin | % Difference |
|---|---|---|---|---|
| Timepoint | Endpoint | (n = 204) | (n = 201) | (95% CI) |
| Early clinical | FDA primary endpoint: | 166 (81.4%) | 162 (80.6%) | 0.8 (−6.9, 8.4) |
| evaluation (ECE) | Cessation of spread, | | | |
| | absence of fever, no | | | |
| | rescue antibiotics | | | |
| | ≧20% reduction of | 190 (93.1%) | 175 (87.1%) | 6.1 (0.5, 11.6)* |
| | lesion area | | | |
| Post therapy | EMA primary endpoint: | 170 (83.3%) | 169 (84.1%) | −0.7 (−7.9, 6.4) |
| evaluation (PTE) | Investigator-assessed | | | |
| | clinical cure | | | |

*p value = 0.032

When the SOLO I and SOLO II studies are combined, the safety profiles were similar across treatment groups (safety population: 976 in oritavancin, 983 in vancomycin). Overall, 55.3% of patients on oritavancin and 56.9% of patients on vancomycin were reported to experience at least one adverse event. Fewer treatment emergent adverse events considered by investigators as related to study drug were reported among patients treated with oritavancin than vancomycin (22.2% vs. 28.4% p=0.002). Proportions of patients with adverse events leading to study drug discontinuation were low and comparable between oritavancin and vancomycin (3.7% and 4.2%, respectively).

While the invention has been described with reference to certain particular embodiments thereof, those skilled in the art will appreciate that various modifications may be made without departing from the spirit and scope of the invention. The scope of the appended claims is not to be limited to the specific embodiments described.

All patents and publications mentioned in this specification are indicative of the level of skill of those skilled in the art to which the invention pertains. Each cited patent and publication is incorporated herein by reference in its entirety.

### CITATIONS

Ambrose P G, Drusano G L, Craig W A. 2012. In vivo activity of oritavancin in animal infection models and rationale for a new dosing regimen in humans. Clin Infect Dis 54 Suppl 3:S220-228.
Arbeit R D, Maki D, Tally F P, Campanaro E, Eisenstein B I; Daptomycin 98-01 and 99-01 Investigators. The safety and efficacy of daptomycin for the treatment of complicated skin and skin-structure infections. Clin Infect Dis. 2004 Jun. 15; 38(12):1673-81.
Arhin et al. 2008. Antimicrob Agents Chemother 52:1597-1603.
Arhin F F, Draghi D C, Pillar, C M, Parr T R Jr, Moeck G, Sahm D F. 2009. Comparative in vitro activity profile of oritavancin against recent gram-positive clinical isolates. Antimicrob. Agents Chemother. 11:4762-4771.
Arhin et al. 2010. Antimicrob Agents Chemother 54:3481-3483.
Ball A T, Xu Y, Sanchez R J, Shelbaya A, Deminski M C, Nau D P. 2010. Nonadherence to oral linezolid after hospitalization: a retrospective claims analysis of the incidence and consequence of claim reversals. Clin Ther. 32(13):2246-55.
Barriere S L. ATLAS trials: efficacy and safety of telavancin compared with vancomycin for the treatment of skin infections. Future Microbiol. 2010 December; 5(12):1765-73.
Belley A, McKay, G A, Beaulie, S, Fadhil I, Arhin F F, Sarmiento I, Parr T R Jr, Moeck G. 2010. Oritavancin disrupts membrane integrity of Staphylococcus aureus and vancomycin resistant enterococci to effect bacterial cell killing. Antimicrob. Agents Chemother. 54:5369-5371.
Belley A, Neesham-Grenon E, McKay G, Arhin F F, Harris R, Beveridge T, Parr T R J. Moeck G. 2009. Oritavancin kills stationary-phase and biofilm Staphylococcus aureus in vitro. Antimicrob. Agents Chemother 53:918-925.
Belley et al. 2013. Antimicrob Agents Chemother 57:205-211.
Breedt J, Teras J, Gardovskis J, Maritz F J, Vaasna T, Ross D P, Gioud-Paquet M, Dartois N, Ellis-Grosse E J, Loh E; Tigecycline 305 cSSSI Study Group. Safety and efficacy of tigecycline in treatment of skin and skin structure infections: results of a double-blind phase 3 comparison study with vancomycin-aztreonam. Antimicrob Agents Chemother. 2005 November; 49(11):4658-66. Centers for Disease Control and Prevention. 2011. Active Bacterial Core Surveillance Report, Emerging Infections Program Network, Methicillin-Resistant Staphylococcus aureus, 2011. Available via the Internet: http://www.cdc.gov/abcs/reports-findings/survreports/mrsall.pdf (Accessed Feb. 25, 2013).
CLSI. 2012a. CLSI document M7-A9.
CLSI. 2012b. CLSI document M100-S22.
Corey G R, Wilcox M H, Talbot G H, Thye D, Friedland D, Baculik T; CANVAS 1 investigators. CANVAS 1: the first Phase III, randomized, double-blind study evaluating ceftaroline fosamil for the treatment of patients with complicated skin and skin structure infections. J Antimicrob Chemother. 2010 November; 65 Suppl 4:iv41-51.
Cubicin prescribing information. Cubist Pharmaceuticals, Inc. January 2013. Accessed online: http://www.cubicin.com/pdf/PrescribingInformation.pdf
Dunbar L M, Milata J, McClure T, Wasilewski M M; SIMPLIFI Study Team. 2011. Comparison of the efficacy and safety of oritavancin front-loaded dosing regimens to daily dosing: an analysis of the SIMPLIFI trial. Antimicrob. Agents Chemother. 55:3476-3484.
Ellis-Grosse E J, Babinchak T, Dartois N, Rose G, Loh E; Tigecycline 300 cSSSI Study Group; Tigecycline 305 cSSSI Study Group. The efficacy and safety of tigecycline in the treatment of skin and skin-structure infections: results of 2 double-blind phase 3 comparison studies with vancomycin-aztreonam. Clin Infect Dis. 2005 Sep. 1; 41 Suppl 5:S341-53.
European Medicines Agency Committee for Medicinal Products for Human Use. Addendum to the note for guidance on evaluation of medicinal products indicated for treatment of bacterial infections (CHMP/EWP/558/95 REV 2) to address indication-specific clinical data (draft). 2012.
http://www.ema.europa.eu/docs/en_GB/document_library/Scientific_guideline/2012/07/WC500 129443.pdf
Food and Drug Administration. Guidance for Industry Acute Bacterial Skin and Skin Structure Infections: Developing Drugs for Treatment (DRAFT GUIDANCE). http://www.fda.gov/downloads/Drugs/GuidanceComplianceRegulatoryInformation/Guidance s/ucm071185.pdf. Accessed 1 December
Hadler J L, Petit S, Mandour M, Cutter M L. Trends in invasive infection with methicillin-resistant Staphylococcus aureus, Connecticut, USA, 2001-2010. Emerg Infect Dis [serial on the Internet]. 2012 June [date cited].
Ito et al. 2012. Antimicrob Agents Chemother 56:4997-4999.
Jauregui L E, Babazadeh S, Seltzer E, Goldberg L, Krievins D, Frederick M, Krause D, Satilovs I, Endzinas Z, Breaux J, O'Riordan W. 2005. Randomized, double-blind comparison of once-weekly dalbavancin versus twice-daily linezolid therapy for the treatment of complicated skin and skin structure infections. Clin Infect Dis. 41(10):1407-15.
Jenkins T C, Sabel A L, Sarcone E E, Price C S, Mehler P S, Burman W J. 2010. Skin and soft-tissue infections requiring hospitalization at an academic medical center: opportunities for antimicrobial stewardship. Clin Infect Dis. 15; 51(8):895-903.
Kumar A, Mann H J, Keshtgarpour M, Flynn M A, Deng H, Far A R, Parr T R, Moriarty S R 2011. In vitro characterization of oritavancin clearance from human blood by low-flux, high-flux, and continuous renal replacement therapy dialyzers. Int J Artif Organs. 34:1067-1074.
Landrum M L, Neumann C, Cook C, Chukwuma U, Ellis M W, Hospenthal D R, Murray C K. Epidemiology of Staphylococcus aureus blood and skin and soft tissue infections in the US military health system, 2005-2010. JAMA, 2012; 308(1): 50-59. doi: 10.1001/jama.2012.7139.
Lee B Y, Singh A, David M Z, Bartsch S M, Slayton R B, Huang S S, Zimmer S M, Potter M A, Macal C M, Lauderdale D S, Miller L G, Daum R S. 2012. The economic burden of community-associated methicillin-resistant Staphylococcus aureus (CA-MRSA). Clin Microbiol Infect. Article first published online: 19 Jun. 2012, DOI: 10.1111/j.1469-0691.2012.03914.x
Liu C, Bayer A, Cosgrove S E, Daum R S, Fridkin S K, Gorwitz R J, Kaplan S L, Karchmer A W, Levine D P, Murray B E, J Rybak M, Talan D A, Chambers H F; Infectious Diseases Society of America. 2011. Clinical practice guidelines by the infectious diseases society of America for the treatment of methicillin-resistant Staphylococcus aureus infections in adults and children. Clin Infect Dis. 52(3):e18-55. doi: 10.1093/cid/ciq146.
McKay G A, Beaulieu S, Arhin F F, Belley A, Sarmiento I, Parr T Jr, Moeck G. Time-kill kinetics of oritavancin and comparator agents against Staphylococcus aureus, Enterococcus faecalis and Enterococcus faecium. J Antimicrob Chemother, 2009; 63: 1191-1199.
Mendes R E, Farrell D J, Sader H S, Jones R N. 2012. Oritavancin microbiologic features and activity results from the surveillance program in the United States. Clin Infect Dis 54 (Suppl 3):S203-5213.
NCCLS. 1999. CLSI document M26-A2.
Noel G J, Bush K, Bagchi P, Ianus J, Strauss R S. A randomized, double-blind trial comparing ceftobiprole medocaril with vancomycin plus ceftazidime for the treatment of patients with complicated skin and skin-structure infections. Clin Infect Dis. 2008 Mar. 1; 46(5):647-55.
Petersen et al. 2013. Clin Microbiol Infec 19:E16-22.
Prokocimer P, De Anda C, Fang E, Mehra P, Das A. Tedizolid phosphate vs linezolid for treatment of acute bacterial skin and skin structure infections: the ESTABLISH-1 randomized trial. JAMA. 2013 Feb. 13; 309(6):559-69.
Rehm S, Campion M, Katz D E, Russo R, Boucher H W. Community-based outpatient parenteral antimicrobial therapy (CoPAT) for Staphylococcus aureus bacteraemia with or without infective endocarditis: analysis of the randomized trial comparing daptomycin with standard therapy. J Antimicrob Chemother. 2009 May; 63(5):1034-42.
Sacchidanand S, Penn R L, Embil J M, Campos M E, Curcio D, Ellis-Grosse E, Loh E, Rose G. Efficacy and safety of tigecycline monotherapy compared with vancomycin plus aztreonam in patients with complicated skin and skin structure infections: Results from a phase 3, randomized, double-blind trial. Int J Infect Dis. 2005 September; 9(5):251-61.
Seltzer E, Don M B, Goldstein B P, Perry M, Dowell J A, Henkel T; Dalbavancin Skin and Soft-Tissue Infection Study Group. Once-weekly dalbavancin versus standard-of-care antimicrobial regimens for treatment of skin and soft-tissue infections. Clin Infect Dis. 2003 Nov. 15; 37(10):1298-303.
Stevens D L, Bisno A L, Chambers H F, Everett E D, Dellinger P, Goldstein E J, Gorbach S L, Hirschmann J V, Kaplan E L, Montoya J G, Wade J C; Infectious Diseases Society of America. 2005. Practice guidelines for the diagnosis and management of skin and soft-tissue infections. Clin Infect Dis 41(10):1373-406.
Stryjewski M E, Graham D R, Wilson S E, O'Riordan W, Young D, Lentnek A, Ross D P, Fowler V G, Hopkins A, Friedland H D, Barriere S L, Kitt M M, Corey G R; Assessment of Telavancin in Complicated Skin and Skin-Structure Infections Study. Telavancin versus vancomycin for the treatment of complicated skin and skin-structure infections caused by gram-positive organisms. Clin Infect Dis. 2008 Jun. 1; 46(11):1683-93.
Talbot G H, Powers J H, Fleming T R, Siuciak J A, Bradley J, Boucher H, CAPB-ABSSSI Project Team. Progress on developing endpoints for registrational clinical trials of community-acquired bacterial pneumonia and acute bacterial skin and skin structure infections: update from the Biomarkers Consortium of the Foundation for the National Institutes of Health. Clin Infect Dis. 2012; 55(8); 1114-1121.
Teflaro prescribing information. Forest Laboratories, Inc. October 2012. Accessed online: http://www.frx.com/pi/teflaro_pi.pdf
Tice A. 2012. Oritavancin: a new opportunity for outpatient therapy of serious infections. Clin Infect Dis S239-243.
Tygacil prescribing information. Pfizer, Inc. November 2012. Accessed online: http://labeling.pfizer.com/showlabeling.aspx?id=491
Vancomycin prescribing information. Pfizer Inc. December 2010. Accessed online: http://labeling.pfizer.com/ShowLabeling.aspx?id=661
Vibativ prescribing information. Theravance Inc. January 2012. Accessed online: http://www.vibativ.com/docs/VIBATIV_PI_Final.pdf
Wilcox M H, Corey G R, Talbot G H, Thye D, Friedland D, Baculik T; CANVAS 2 investigators. CANVAS 2: the second Phase III, randomized, double-blind study evaluating ceftaroline fosamil for the treatment of patients with complicated skin and skin structure infections. J Antimicrob Chemother. 2010 November; 65 Suppl 4:iv53-iv65.
Wilson S E, O'Riordan W, Hopkins A, Friedland H D, Barriere S L, Kitt M M; ATLAS Investigators. Telavancin versus vancomycin for the treatment of complicated skin and skin-structure infections associated with surgical procedures. Am J Surg. 2009 June; 197(6):791-6.
Zhanel G G, Schweizer F, Karlowsky J A. 2012. Oritavancin:mechanism of action. Clin Infect Dis 54 (Suppl 3):S214-S219.
Zyvox prescribing information. Pfizer Inc. March 2012. Accessed online: http://labeling.pfizer.com/showlabeling.aspx?id=649.

1-50. (canceled)

51. A method of treating or preventing a skin lesion in a subject, comprising administering a therapeutically effective amount of a pharmaceutical composition comprising oritavancin or a pharmaceutically acceptable salt thereof to a subject having a skin lesion or at risk of developing a skin lesion, thereby treating or preventing a skin lesion in a subject.

52. The method of claim 51, wherein the bacteria causing the lesion is a Gram-positive bacteria.

53. The method of claim 51, wherein the bacteria causing the lesion is one or more of Staphylococcus aureus, methicillin-susceptible Staphylococcus aureus (MSSA), methicillin-resistant Staphylococcus aureus (MRSA), a multi-drug resistant (MDR) strain of MSSA, a MDR strain of MRSA, and a mecC-expressing strain of MRSA.

54. The method of claim 51, wherein the bacteria causing the lesion is one or more bacteria selected from the group consisting of Staphylococcus aureus, vancomycin-resistant Staphylococcus aureus, vancomycin-intermediate Staphylococcus aureus, vancomycin hetero-intermediate Staphylococcus aureus, Streptococcus pyogenes, Streptococcus agalactiae, Streptococcus anginosus, Streptococcus intermedius, Streptococcus constellatus, Streptococcus dysgalactiae, Streptococcus dysgalactiae subsp. equisimilis, Streptococci Group C, F and G species, Staphylococcus lugdunensis, Enterococcus faecalis, vancomycin-resistant Enterococcus faecalis, Enterococcus faecium, and vancomycin-resistant Enterococcus faecium.

55. The method of claim 51, wherein the treatment achieves a cessation of an increase in surface area of the lesion within about 24 hours of the administering.

56. The method of claim 51, wherein the treatment achieves a prevention in increase in the surface area of the lesion within 24 hours of the administering.

57. The method of claim 51, wherein the treatment achieves a reduction in surface area of the lesion of at least about 20% within about 48 hours of the administering.

58. The method of claim 51, wherein the treatment achieves a reduction in fever in the subject within about 12 hours of the administering.

59. The method of claim 51, wherein the pharmaceutical composition comprises at least about 1200 mg of oritavancin or a salt thereof.

60. The method of claim 51, wherein treatment or prevention is achieved by administering to the subject a single dose of a pharmaceutical composition comprising about 1200 mg of oritavancin or a salt thereof.

61. The method of claim 51, wherein said administering is via intravenous administration.

62. The method of claim 51, wherein said skin lesion is a lesion selected from the group consisting of ulcer, macule, vesicle, pustule, papule, nodule, wheal, and telangiectasia.

63. A method of reducing the size of a skin lesion in a subject, comprising administering a therapeutically effective amount of a pharmaceutical composition comprising oritavancin or a pharmaceutically acceptable salt thereof to a subject having a skin lesion, thereby reducing the size of a skin lesion in a subject.

64. The method of claim 63, wherein the bacteria causing the lesion is a Gram-positive bacteria.

65. The method of claim 63, wherein the bacteria causing the lesion is one or more of Staphylococcus aureus, methicillin-susceptible Staphylococcus aureus (MSSA), methicillin-resistant Staphylococcus aureus (MRSA), a multi-drug resistant (MDR) strain of MSSA, a MDR strain of MRSA, and a mecC-expressing strain of MRSA.

66. The method of claim 63, wherein the bacteria causing the lesion is one or more bacteria selected from the group consisting of Staphylococcus aureus, vancomycin-resistant Staphylococcus aureus, vancomycin-intermediate Staphylococcus aureus, vancomycin hetero-intermediate Staphylococcus aureus, Streptococcus pyogenes, Streptococcus agalactiae, Streptococcus anginosus, Streptococcus intermedius, Streptococcus constellatus, Streptococcus dysgalactiae, Streptococcus dysgalactiae subsp. equisimilis, Streptococci Group C, F and G species, Staphylococcus lugdunensis, Enterococcus faecalis, vancomycin-resistant Enterococcus faecalis, Enterococcus faecium, and vancomycin-resistant Enterococcus faecium.

67. The method of claim 63, wherein the administration achieves a reduction in surface area of the lesion within about 24 hours of the administering.

68. The method of claim 63, wherein the administration achieves a reduction in surface area of the lesion of at least about 20% within about 48 hours of the administering.

69. The method of claim 63, wherein the pharmaceutical composition comprises at least about 1200 mg of oritavancin or a salt thereof.

70. The method of claim 63, wherein reduction in size is achieved by administering to the subject a single dose of a pharmaceutical composition comprising about 1200 mg of oritavancin or a salt thereof.

71. The method of claim 63, wherein said administering is via intravenous administration.

72-96. (canceled)
